SEQUENCE LISTING (18 sequences)

SEQ ID NO: 1 - protein, 5 aa, Artificial
  Synthetic ELP repeat
  MISC_FEATURE (4)..(4): X can be any amino acid except proline
VPGXG

SEQ ID NO: 2 - protein, 160 aa, Artificial
  Synthetic ELP
VGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPG

SEQ ID NO: 3 - protein, 400 aa, Artificial
  Synthetic ELP
VGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPG

SEQ ID NO: 4 - protein, 800 aa, Artificial
  Synthetic ELP
VGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPG

SEQ ID NO: 5 - protein, 200 aa, Artificial
  Synthetic ELP
GGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPG

SEQ ID NO: 6 - protein, 400 aa, Artificial
  Synthetic ELP
GGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPG

SEQ ID NO: 7 - protein, 800 aa, Artificial
  Synthetic ELP
GGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPGGGVPG

SEQ ID NO: 8 - protein, 160 aa, Artificial
  Synthetic ELP
VGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPG

SEQ ID NO: 9 - protein, 400 aa, Artificial
  Synthetic ELP
VGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPG

SEQ ID NO: 10 - protein, 800 aa, Artificial
  Synthetic ELP
VGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPGVGVPGAGVPGGGVPGAGVPGGGVPGAGVPGGGVPGAGVPG

SEQ ID NO: 11 - protein, 200 aa, Artificial
  Synthetic ELP
KGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPG

SEQ ID NO: 12 - protein, 400 aa, Artificial
  Synthetic ELP
KGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPG

SEQ ID NO: 13 - protein, 800 aa, Artificial
  Synthetic ELP
KGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPGKGVPG

SEQ ID NO: 14 - protein, 932 aa, Artificial
  Synthetic ELP-VEGF
MSKGPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGWPGSGAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 15 - protein, 840 aa, Artificial
  Synthetic SynB1-ELP-p50
MRGGRLSYSRRRFSTSTGRGCGPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGWPGSGGVQRKRQKLMP

SEQ ID NO: 16 - protein, 839 aa, Artificial
  Synthetic SynB1-ELP-NOX
MRGGRLSYSRRRFSTSTGRGCGPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGWPGSGGCSTRIRRQL

SEQ ID NO: 17 - protein, 10 aa, Artificial
  NF-kB inhibitory peptide
VQRKRQKLMP

SEQ ID NO: 18 - protein, 9 aa, Artificial
  NADPH oxidase inhibitory peptide
CSTRIRRQL

(19) United States
(12) Patent Application Publication
(10) Pub. No.: US 2020/0362016 A1
(43) Pub. Date: Nov. 19, 2020

(54) Molecular Size of Elastin-Like Polypeptide Delivery System for Therapeutics Modulates Deposition in the Placenta

(71) Applicant: University of Mississippi Medical Center, Jackson, MS (US)

(72) Inventors: Gene L. Bidwell, III, Brandon, MS (US); Eric M. George, Jackson, MS (US)

(21) Appl. No.: 16/835,041

(22) Filed: Mar. 30, 2020

Related U.S. Application Data
(63) Continuation-in-part of application No. 16/104,037, filed on Aug. 16, 2018.
Application No. 16/104,037 is a continuation of application No. 14/917,460, filed on Mar. 8, 2016, now Pat. No. 10,081,667.
(60) Provisional application No. 62/826,447, filed on Mar. 29, 2019.
(60) Provisional application No. 61/885,300, filed on Oct. 1, 2013.

(51) Int. Cl. C07K 14/78; A61K 38/18; A61K 38/00; A61K 47/64; C07K 7/08; C07K 14/47; C07K 14/49

(52) U.S. Cl. CPC C07K 14/78; A61K 38/1866; A61K 38/005; A61K 47/6435; C07K 2319/33; C07K 14/4703; C07K 14/49; C07K 2319/00; C07K 7/08

(57) ABSTRACT

A composition including an elastin-like polypeptide (ELP) coupled to a therapeutic agent is provided. The ELP comprises at least about 5 repeats of the amino acid sequence VPGXG. Further provided is a method of using the composition for therapeutic agent delivery during pregnancy to reduce the amount of the therapeutic agent crossing a placenta in a pregnant subject. The method includes administering to the pregnant subject an effective amount of the composition comprising the ELP coupled to the therapeutic agent.

### RELATED APPLICATIONS

This application claims priority to U.S. Provisional Application Ser. No. 62/826,447, filed Mar. 29, 2019, and is a continuation-in-part of U.S. patent application Ser. No. 16/104,037, filed Aug. 16, 2018, which claims priority from U.S. patent application Ser. No. 14/917,460, filed Mar. 8, 2016, which is a national phase application of International Patent Application No. PCT/US2014/058640, filed Oct. 1, 2014, which claims priority to U.S. Provisional Application Ser. No. 61/885,300, filed Oct. 1, 2013, the entire disclosures of which are incorporated herein by this reference.

### STATEMENT OF GOVERNMENT SUPPORT

This presently-disclosed subject matter was made with government support under grant numbers NIH R01HL121527 and R01HL137791 awarded by National Institutes of Health. The government has certain rights in it.

### SEQUENCE LISTING

The instant application contains a Sequence Listing which has been submitted in ASCII format via EFS-Web and is hereby incorporated by reference in its entirety. The ASCII copy of the Sequence Listing, which was created on Aug. 5, 2020, is named 11637N-181022.txt and is 70.1 kilobytes in size.

### TECHNICAL FIELD

The presently-disclosed subject matter relates to articles and methods for targeted delivery of therapeutic during pregnancy. More particularly, the presently-disclosed subject matter relates to an elastin-like polypeptide (ELP) and methods of use thereof for targeted delivery of therapeutic agents to the placenta in a pregnant subject.

### INTRODUCTION

In the U.S. alone, there were 3.98 million births reported in 2015. According to the FDA, at least 50% of these women took at least one medication during pregnancy. However, special considerations must be taken when giving drug therapies to pregnant mothers. Not only must normal concerns of maximizing efficacy while reducing side effects in such subjects be considered, but the effects of the therapeutic agent on the developing fetus must also be taken into account. Many therapeutic agents that are otherwise safe for an adult will cross the placental barrier in pregnant mothers and cause severe adverse effects on the developing fetus.

In view thereof, the ability of different therapeutic agents to cross the placental barrier has been investigated, although the results have been highly variable. For example, while many large molecules such as proteins are prevented from passively crossing the placental barrier, some proteins, such as immunoglobulins, are actively transported across the placental barrier via the neonatal Fc receptor, FcRn, expressed on syncytiotrophoblasts. Transferrin also has receptors on trophoblasts and is actively transported across the placental barrier. The investigation of some high molecular weight (MW) drug carriers for placental transfer has similarly resulted in highly variable results. That is, some types of nanoparticles readily cross the placenta and some do not. Taken as a whole, these studies show that placental transfer isn't restricted by merely the size of macromolecular drug carriers, but is also dependent on the hydrophobicity and charge of the therapeutic agents.

Despite the uncertainty with respect to placental transfer, many of those drugs had no clinical trials in pregnant women. Moreover, pregnancy is often an exclusion criterion for clinical trials due to possible deleterious effects to the fetus. Drug development is additionally hindered because of risk aversion from the pharmaceutical industry and from regulatory bodies. The current strategy for evaluating the safety of drugs that might be used during pregnancy requires initial reproductive toxicity testing in the preclinical and early clinical phase and post-approval monitoring. During preclinical development, studies of developmental and reproductive toxicology (DART) are typically done in mice, rats and rabbits. Additionally, post-approval monitoring in pregnant subjects for drugs not restricted for use during pregnancy is often carried out as part of Phase IV studies. Still, actual patient data on effects of drugs during pregnancy is often scarce. Furthermore, direct drug development for adverse pregnancy-specific conditions is extremely limited. Nevertheless, the need to administer drugs during pregnancy, especially drugs for treatment of pregnancy-related conditions remains high.

Therefore, compositions and methods that reduce the amount of therapeutic agents crossing the placenta in a pregnant subject and which can be used to treat various diseases and disorders during pregnancy are both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and, in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a placental region targeting elastin-like polypeptide (ELP) including between 5 and 671 repeat units having the sequence VPGXG, where X in each of the repeat units is individually selected from the group consisting of any amino acid except proline, and where the placental region targeting ELP is selected from the group consisting of a chorionic plate targeting ELP and a chorionic plate and labyrinth/junctional zones targeting ELP. In some embodiments, the ELP is a chorionic plate targeting ELP. In some embodiments, the ELP comprises up to 70 of the repeat units. In one embodiment, the ELP comprises between 5 and 70 of the repeat units. In some embodiments, the ELP comprises a molecular weight of up to 30 kDa. In one embodiment, the ELP comprises a molecular weight of between 3 kDa and 30 kDa. In some embodiments, at least 90% of the ELP accumulates in the chorionic plate.

In some embodiments, the ELP is a chorionic plate and labyrinth/junctional zones targeting ELP. In some embodiments, the ELP comprises at least 95 of the repeat units. In one embodiment, the ELP comprises between 95 and 671 of the repeat units. In some embodiments, the ELP comprises a molecular weight of at least 37 kDa. In one embodiment, the ELP comprises a molecular weight of between 37 kDa and 257 kDa. In some embodiments, at least 15% of the ELP accumulates in labyrinth/junctional zones of the placenta.

In some embodiments, the repeat units include V:G:A in a 1:4:3 ratio. In some embodiments, the ELP further comprising one or more of a group selected from a therapeutic agent or agents, a drug binding domain, a targeting domain, and a cell penetrating peptide.

Also provided herein, in some embodiments, is a method of treating a disease in a pregnant subject, the method including administering a placental region targeting elastin-like peptide (ELP) and a therapeutic drug to a subject in need thereof; where the ELP includes between 5 and 700 repeat units having the sequence VPGXG (SEQ ID NO: 1), where X in each of the repeat units is individually selected from the group consisting of any amino acid except proline; and where the placental region targeting ELP is selected from the group consisting of a chorionic plate targeting ELP, a chorionic plate and labyrinth/junctional zones targeting ELP, and a primarily labyrinth/junctional zones targeting ELP. In some embodiments, the placental region targeting ELP further comprises one or more of a group selected from a drug binding domain, a targeting domain, and a cell penetrating peptide.

Further provided herein, in some embodiments, is a method for decreasing the rate of clearance of an elastin-like polypeptide (ELP) from plasma or a tissue, the method including increasing the number of repeat units in the ELP.

Further features and advantages of the presently-disclosed subject matter will become evident to those of ordinary skill in the art after a study of the description, figures, and non-limiting examples in this document.

### BRIEF DESCRIPTION OF THE DRAWINGS

FIG. 1 includes schematic diagrams showing some of the exemplary compositions described herein, including: a schematic diagram showing an Elastin-like polypeptide (ELP) drug carrier fused to VEGF₁₂₁; an ELP carrier fused to a peptide inhibitor of the NF-κB pathway; and an ELP carrier fused to a peptide inhibitor of NADPH oxidase. The polypeptides also contain a cell penetrating peptide to mediate uptake into target cells.

FIGS. 2A-B include a series of images and a graph showing the biodistribution of ELP in pregnant rats. (A) The ELP drug carrier was labeled with AlexalFluor633 and infused into normal pregnant Sprague Dawley rats by IV injection (100 mg/kg). 4 h after injection, ex vivo images of placentas, pups, and major organs were collected using an IVIS Spectrum. (B). Fluorescence intensity was quantified in all tissues. Error bars represent the standard deviation of 3 rats.

FIGS. 3A-D include a series of images and a graph illustrating placental distribution of ELP and SynB1-ELP. (A) After IV administration of fluorescently-labeled polypeptides or saline control, placentas, pups, and major organs were removed and imaged ex vivo using an IVIS Spectrum. Polypeptide deposition in placentas and pups is shown. (B) Fluorescence levels in placentas, pups, and major organs was quantified using Living Image software. Error bars represent the standard deviation of four rats per treatment group. (C) Frozen sections of intact placentas and pups were cut, stained with the actin-specific rhodamine-phalloidin to allow visualization of the placenta and pups, and scanned using a florescence slide scanner. (D) The same slides were imaged using a florescence microscope to visualize the cellular localization of the polypeptides in the placenta (100× magnification).

FIGS. 4A-E include graphs and images illustrating dye release and protein stability in plasma. (A) Rhodamine-labeled ELP and SynB1-ELP were incubated in plasma from pregnant rats for the indicated time at 37° C. Percentage of dye release is shown for an average of two experiments, bars indicate s.d. (B-E) In vivo protein stability was determined by SDS-PAGE analysis of plasma samples from the pharmacokinetic experiment. A representative gel from one animal in ELP (B) and SynB1-ELP (D) is shown. The numbers indicate time points, and the final lane was loaded with 10 mg of the injected protein as a loading control. The total band intensity and % <50 kDa are shown for an average of four animals per group in ELP (C) and SynB1-ELP (E), bars indicate s.d.

FIGS. 5A-B include image and bar graph showing Quantitative Fluorescence Histology of Feto-amnio-placental Units. Frozen feto-amnio-placental units were cut to 20 mm sections, and slides were scanned with a fluorescence slide scanner. (A) Representative images from each animal were collected with identical scan settings. (B) Data from all slide scans were quantified relative to fluorescence standards made from the injected protein cut to the same thickness. * Statistically significant as determined by a t-test (p<0.05). ** Levels were not detectable over autofluorescence.

FIGS. 6A-B include images illustrating intra-placental distribution of ELPs. (A-B) Slides of cryosections were immunostained with a cytokeratin antibody to mark trophoblast cells (green), and fluorescence of the rhodamine-labeled ELP (A) and SynB1-ELP (B) was detected (red). The 40× magnification shows polypeptide accumulation at the chorionic plate (solid arrows) and in the labyrinth. The 100× magnification shows polypeptide in the cytoplasm of trophoblast cells but excluded from the fetal chorionic villi (open arrows).

FIGS. 7A-C include images and graphs showing plasma levels and biodistribution of ELPs after Chronic Infusion. (A) Rhodamine-labeled ELP or SynB1-ELP was administered chronically by IP minipump from GD14-GD19. Plasma was sampled throughout the experiment, and polypeptide levels were determined relative to standards of the injected protein. Data represent the mean±s.d. of four rats per group. (B) Ex vivo fluorescence imaging of eight pups and corresponding placentas from one rat from each group is shown. (C) Fluorescence intensities were quantified, corrected for autofluorescence, and fit to standards of the injected proteins. Data represent the mean±s.e. of eight placentas and eight pups per rat and four rats per group. * Statistically significant as determined by a two-way ANOVA with post-hoc Bonferroni multiple comparisons (p<0.05). ** Levels were not detectable over autofluorescence.

FIG. 8 is an image of a gel showing the purification of ELP-VEGF. SDS-PAGE gel with silver staining demonstrates the purity of ELP-VEGF and ELP control polypeptides. Lane 1, ELP; Lane 2, ELP-VEGF₁₂₁; Lane 3, VEGF₁₂₁.

FIG. 9 is a bar graph showing ELP-VEGF stimulation of HUVEC proliferation. HUVEC cell proliferation was determined after 72 h exposure to ELP, VEGF, or ELP-VEGF at the indicated concentrations using the MTS cell proliferation assay.

FIGS. 10A-B include a series of images and a bar graph showing ELP-VEGF stimulation of tube formation in HUVECs. (A) HUVEC tube formation was assessed 6 h after seeding on growth factor reduced Matrigel and supplementing the media with 20 nM ELP, VEGF, or ELP-VEGF. (B) Average tubes per field were counted for six fields per sample. Data represent the mean±se of four independent experiments. * p<0.01. one way ANOVA with post-hoc Bonferonni multiple comparison.

FIGS. 11A-B include a bar graph and a series of images illustrating the ELP-VEGF stimulation of HUVEC migration. (A) HUVEC migration was assessed 16 h after seeding in the top chamber of Matrigel-coated Boyden chambers in minimal media and supplementing the bottom chamber with minimal media plus ELP, VEGF, or ELP-VEGF at the indicated concentrations. (B) Average cells per field were counted for four to seven fields per sample. Data represent the mean±se of three independent experiments. * p<0.01, one way ANOVA with post-hoc Bonferonni multiple comparison.

FIGS. 12A-D include graphs showing ELP-VEGF pharmacokinetics and biodistribution. (A) Fluorescently labeled free VEGF or ELP-VEGF were administered by IV injection to C57/B16 mice. Plasma levels were determined by direct fluorescence quantitation and fit to a two-compartment pharmacokinetic model. (B-C) ELP-VEGF had a significantly lower plasma clearance rate than free VEGF (B), as was evidenced by lower levels in the urine at the end of the experiment (C) Data represent the mean±sd of four mice per group. * p<0.01, Student's t-test. (D) ELP fusion significantly altered the biodistribution of VEGF, increasing its levels in the spleen and liver and reducing its levels in the kidney. * p<0.01, one way ANOVA with post-hoc Bonferroni multiple comparison.

FIG. 13 is a bar graph showing effect of ELP-VEGF on blood pressure in the reduced uterine perfusion model. A study was conducted in pregnant rats subjected to surgical reduction of uterine blood flow (RUPP) on gestational day 14 (GD14). ELP-VEGF or saline control was administered by continuous infusion via IP minipump from GD14 to GD19 at a dose of 1 mg/kg/day. Data represent the mean arterial pressure as assessed by indwelling carotid catheters on GD19. Individual animal data are indicated by the points.

FIGS. 14A-B include images and a bar graph illustrating inhibition of NF-κB activation with an ELP-delivered p50 peptide. (A) Localization of NF-κB in HUVEC cells before and after TNFα stimulation. HUVECs were treated with SynB1-ELP control or SynB1-ELP-p50 (20 μM) for 24 h. Cells were then stimulated for 1 h with TNFα, and NF-κB localization was determined by immunostaining for the p65 subunit (green) and for nuclei with DAPI (blue). (B) The nuclear: cytoplasmic ratio of NF-κB staining was determined under each treatment condition. Data represent the mean of 30-60 cells per treatment and are averaged over three experiments.

FIG. 15 includes a graph illustrating the inhibition of endothelin release by SynB1-ELP-p50. HUVECs growing in 24-well plates were treated with 50 μM SynB1-ELP or SynB1-ELP-p50 overnight, then 50 or 100 ng/mL was added. Cells were incubated overnight, and culture media was collected and frozen. Endothelin-1 concentration was determined by ELISA. * Levels are significantly different from untreated HUVECs. ** Levels are significantly reduced relative to TNFα treatment only (p<0.01, one way ANOVA with post-hoc Bonferonni multiple comparison).

FIGS. 16A-B include graphs illustrating that the ELP-delivered NF-κB inhibitory peptide is not toxic to cells. (A-B) HUVEC endothelial cells (A) and BeWo chorionic cells (B) were exposed to the indicated concentration of SynB1-ELP or SynB1-ELP-p50 for 72 h. Cell number was determined by MTS assay. Bars represent the standard error of the mean of five independent experiments.

FIGS. 17A-B include graphs and images illustrating pharmacokinetics and biodistribution of the ELP-delivered p50 peptide. Free p50 peptide (10 amino acids) was synthesized with an N-terminal rhodamine label. Also, the SynB1-ELP-fused p50 peptide was purified and labeled with rhodamine. Each agent was administered by IV injection into pregnant rats at GD14. (A) Plasma was sampled over time, and peptide levels were determined by quantitative fluorescence analysis. (B) Four hours after injection, placenta and pup levels were determined by ex vivo whole organ fluorescence imaging.

FIG. 18 includes images showing the cellular internalization of SynB1-ELP delivered NOX peptide. Rhodamine-labeled SynB1-ELP-NOX was exposed to cells for 1 h, cells were washed, fresh media was returned, and images were collected 24 h after initial exposure.

FIG. 19 includes a graph showing inhibition of reactive oxygen species production of placental villous explants. Villous explants were dissected from rat placentas at day 19 of gestation. Explants were grown ex vivo on a bed of Matrigel in cell culture medium. 24 h after equilibration of explants, media was replaced with media plus SynB1-ELP or SynB1-ELP-NOX, and explants were incubated at 37° C. in a 6% or 1% O₂ environment for 48 h. ROS was detected by incubation with 5 μM dihydroethidium for 1 hour, and fluorescence was measured using a florescence plate reader. The assay was performed in triplicate, and data represent the mean±s.e. of three independent experiments each performed with 4 explants per treatment group. *p<0.01, one way ANOVA with post-hoc Bonferonni multiple comparison.

FIGS. 20A-D include images and bar graphs illustrating enhancement of kidney specificity using kidney targeting peptides. (A) Rats were administered fluorescently labeled ELP, SynB1-ELP, Tat-ELP, or KTP-ELP, and organ biodistribution was determined by ex vivo fluorescence imaging. (B) Quantitative analysis showed that the highest accumulation of all peptides was in the kidney, and the targeting agents significantly increased kidney deposition. (C-D) KTP-ELP had the highest specificity for the kidney as assessed by kidney:liver (C) and kidney:heart (D) ratios.

FIGS. 21A-B show graphs illustrating plasma pharmacokinetics of ELP constructs in a rat pregnancy model. (A) Direct fluorescence measurement of plasma shows an increase in MW of ELPs resulted in slower plasma clearance. (B) Plasma fluorescence was measured before and after precipitation of the proteins with TCA to assess the effects of fluorophore loss from polypeptides.

FIGS. 22A-B show representative ex vivo whole organ florescence images of major organs, placenta, and pups after bolus i.v. injection using ELP proteins with varying MW. (A) Representative image of major organs from one animal from each group. (B) Representative image of placentae and corresponding pups from one animal from each group.

FIGS. 23A-E show graphs illustrating biodistribution of ELP proteins having varying MW a rat pregnancy model. (A) Tissue accumulation of ELP-63, ELP-127, and ELP-223 in each of the brain, lungs, spleen, kidney, liver, heart, placenta, and pups. (B) Accumulation of ELPs in the brain based upon molecular weight. (C) Accumulation of ELPs in the kidney based upon molecular weight. (D) Accumulation of ELPs in the liver based upon molecular weight. (E) Accumulation of ELPs in the placenta based upon molecular weight.

FIGS. 24A-D show graphs and images illustrating levels of rhodamine in the urine for various ELPs. (A) Rhodamine levels in the urine corrected by creatinine. (B) Percent free fluorophore in the urine as determined by fluorescence analysis before and after TCA precipitation. (C-D) SDS-PAGE analysis of urine samples with direct fluorescence imaging (C) and Coomassie staining (D).

FIG. 25 shows an image illustrating placental distribution of ELP constructs.

FIG. 26 shows a graph illustrating levels of various ELPs in the chorionic plate and labyrinth/junctional regions of the placenta.

### BRIEF DESCRIPTION OF THE SEQUENCE LISTING

SEQ ID NO: 1 is a ELP amino acid sequence VPGXG, where X can be any amino acid except proline.

SEQ ID NO: 2 is a ELP sequence of 32 repeats of the amino acid sequence VPGXG, where X is Val, Ala, and Gly in a 1:8:7 ratio

SEQ ID NO: 3 is a ELP sequence of 80 repeats of the amino acid sequence VPGXG, where X is Val, Ala, and Gly in a 1:8:7 ratio.

SEQ ID NO: 4 is a ELP sequence of 160 repeats of the amino acid sequence VPGXG, where X is Val, Ala, and Gly in a 1:8:7 ratio.

SEQ ID NO: 5 is a ELP sequence of 40 repeats of the amino acid sequence VPGXG, where X is Gly.

SEQ ID NO: 6 is a ELP sequence of 80 repeats of the amino acid sequence VPGXG, where X is Gly.

SEQ ID NO: 7 is a ELP sequence of 160 repeats of the amino acid sequence VPGXG, where X is Gly.

SEQ ID NO: 8 is a ELP sequence of 32 repeats of the amino acid sequence VPGXG, where X is Val, Ala, or Gly in a 1:4:3 ratio.

SEQ ID NO: 9 is a ELP sequence of 80 repeats of the amino acid sequence VPGXG, where X is Val, Ala, or Gly in a 1:4:3 ratio.

SEQ ID NO: 10 is a ELP sequence of 160 repeats of the amino acid sequence VPGXG, where X is Val, Ala, or Gly in a 1:4:3 ratio.

SEQ ID NO: 11 is a ELP sequence of 40 repeats of the amino acid sequence VPGXG, where X is Lys.

SEQ ID NO: 12 of a ELP sequence of 80 repeats of the amino acid sequence VPGXG, where X is Lys.

SEQ ID NO: 13 is a ELP sequence of 160 repeats of the amino acid sequence VPGXG, where X is Lys.

SEQ ID NO: 14 is a ELP-VEGF amino acid sequence, where a ELP sequence (SEQ ID NO: 4) fused to a C-terminal VEGF121 sequence.

SEQ ID NO: 15 is a SynB1-ELP-p50 amino acid sequence, where a SynB1 peptide fused to N-terminus of a ELP sequence (SEQ ID NO: 4), and a p50 peptide sequence fused to the C-terminus of the ELP sequence.

SEQ ID NO: 16 is a SynB1-ELP-NOX amino acid sequence, where a SynB1 peptide sequence fused to the N-terminus of a ELP sequence (SEQ ID NO: 4), and NOX peptide fused to the C-terminus of the ELP sequence.

SEQ ID NO: 17 is a NF-κB inhibitory peptide amino acid sequence.

SEQ ID NO: 18 is a NADPH oxidase inhibitory peptide amino acid sequence.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. Further, while the terms used herein are believed to be well-understood by one of ordinary skill in the art, definitions are set forth to facilitate explanation of the presently-disclosed subject matter.

Some of the polynucleotide and polypeptide sequences disclosed herein are cross-referenced to GENBANK® accession numbers. The sequences cross-referenced in the GENBANK® database are expressly incorporated by reference as are equivalent and related sequences present in GENBANK® or other public databases. Also expressly incorporated herein by reference are all annotations present in the GENBANK® database associated with the sequences disclosed herein. Unless otherwise indicated or apparent, the references to the GENBANK® database are references to the most recent version of the database as of the filing date of this Application.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

The presently-disclosed subject matter relates to compositions and methods for therapeutic agent delivery during pregnancy. More particularly, the presently-disclosed subject matter relates to elastin-like polypeptides (ELPs), a composition comprising an elastin-like polypeptide (ELP) coupled to a therapeutic agent, and a method of using the composition to reduce the amount of the therapeutic agent crossing the placenta in a pregnant subject. As used herein, the term “elastin-like polypeptide” or “ELP” refers to a synthetic, or genetically engineered, protein containing structural peptide units, which may be repeating units, structurally related to, or derived from, sequences of the elastin protein.

In some embodiments, the ELP includes at least about 5 repeats of the amino acid sequence VPGXG (SEQ ID NO: 1). In some embodiments, the ELP includes up to about 671 repeats of SEQ ID NO: 1 (Table 1). In one embodiment, for example, the ELP sequences comprises about 5 repeats to about 671 repeats of SEQ ID NO: 1. The X position in each repeat of SEQ ID NO: 1 individually includes any amino acid except proline. For example, in one embodiment, the X position in each repeat of SEQ ID NO: 1 includes V, A, or G. In another embodiment, the X in SEQ ID NO: 1 is V, A, and Gin a ratio of 1:4-8:3-7.

TABLE 1
ELP constructs, their coding sequence
size, and predicted protein MW.

| | Number of VPGxG | | Number of | Predicted |
|---|---|---|---|---|
| | (SEQ ID NO: 1) | Insert size | amino acid | protein MW |
| Protein | repeats | (bp) | residues | (kDa) |
| ELP-31 | 31 | 480 | 170 | 13.0977 |
| ELP-63 | 63 | 960 | 330 | 25.2475 |
| ELP-95 | 95 | 1440 | 490 | 37.3972 |
| ELP-127 | 127 | 1920 | 650 | 49.5469 |
| ELP-159 | 159 | 2400 | 810 | 61.696 |
| ELP-191 | 191 | 2880 | 970 | 73.8463 |
| ELP-223 | 223 | 3360 | 1130 | 85.996 |
| ELP-255 | 255 | 3840 | 1290 | 98.1457 |
| ELP-287 | 287 | 4320 | 1450 | 110.2955 |
| ELP-351 | 351 | 5280 | 1770 | 122.4452 |
| ELP-415 | 415 | 6240 | 2090 | 158.8943 |
| ELP-479 | 479 | 7200 | 2410 | 183.1937 |
| ELP-543 | 543 | 8160 | 2730 | 207.4932 |
| ELP-671 | 671 | 10080 | 3370 | 256.092 |

Since ELPs are genetically engineered rather than chemically synthesized, the sequence and molecular weight thereof can be precisely controlled. As such, the composition and/or length of the ELP sequence may be modified through know methods, such as, but not limited to, recursive directional ligation. For example, in some embodiments, the composition and/or length of the ELP sequence may be modified to include therapeutic proteins or peptides, targeting proteins or peptides, cell penetrating peptides, reactive sites for chemical attachment of therapeutic agents, or a combination thereof. These modified ELPs form inert and biodegradable macromolecule carriers that have good pharmacokinetic profiles, very low immunogenicity, and can stabilize small proteins, small peptides, and/or small molecule therapeutic agent cargo in systemic circulation. Accordingly, when used as a delivery system for therapeutics, the ELPs disclosed herein provide certain therapeutic advantages to the therapeutic agent(s), such as, but not limited to, comparatively better stability, solubility, bioavailability, half-life, persistence, biological action of the therapeutic proteinaceous component or attached small molecule drug.

In some embodiments, the ELP includes a drug binding domain in place of or in addition to the fused and/or chemically attached therapeutic agent. The drug binding domain facilitates attachment of any suitable known or new small molecule therapeutic agent(s). In some embodiments, the drug binding domain is attached to the ELP carrier via a drug release domain to allow for selective release of the drug under particular environmental conditions or at specific sites within the body. In some embodiments, the drug binding domain improves delivery of the therapeutic agent. For example, the drug binding domain may improve the delivery of therapeutic agents to treat preeclampsia and other pregnancy related disorders, or to treat other diseases that happen to occur during pregnancy such as cancer. Additionally or alternatively, in some embodiments, the ELP coupled therapeutic system includes multiple copies of the therapeutic agent and/or drug binding domain to increase the amount of drug delivered. This may also include the use of two or more different therapeutic agents or different drugs attached to the ELP and/or drug binding domain(s) to achieve combination therapy. Other cases may include both a therapeutic agent/s and a drug binding domain/s to achieve simultaneous delivery of peptide/protein—based therapeutic agents with small molecule drugs.

Furthermore, as opposed to chemically synthesized polymers, the genetically engineered ELPs and ELP-fusion proteins disclosed herein may be expressed in E. coli or other recombinant expression systems. In some embodiments, this facilitates easy production and/or purification of large quantities of the molecules. For example, in some embodiments, the ELPs including the repeats of SEQ ID NO: 1 have a unique physical property called thermal responsiveness, where the polypeptide forms aggregates above a characteristic transition temperature and the aggregates re-dissolve below the transition temperature. When a lysate containing such recombinantly expressed ELPs is heated above the polypeptides' transition temperature the ELPs aggregate. These aggregated ELPs are then collected by centrifugation. Repeated centrifugation above and below the transition temperature leads to large quantities of very pure protein. As will be appreciated by those skilled in the art, the composition and/or length of the ELP sequence may be modified to influence the ELP's transition temperature (Tₜ), further facilitating ease of purification.

It has now been determined, that ELP does not cross the placenta, and that it can be used as a carrier for therapeutic peptides, antibiotics, and small molecule drugs in a manner that allows pregnant mothers to be treated with a therapeutic agent, while the amount of therapeutic agent crossing the placenta is reduced to thereby protect the developing fetus from damage by the therapeutic agent. Thus, in some embodiments of the presently-disclosed subject matter, the ELP is a therapeutic agent delivery vector that does not cross the placental barrier. As described in further detail below, this therapeutic agent delivery vector is capable of fusion to many types of therapeutic agents, including small molecules, antibiotics, therapeutic peptides, therapeutic proteins, and nucleic acids and allows those therapeutic agents to be stabilized in the maternal circulation, while also preventing them from entering the fetal circulation.

In some embodiments of the presently-disclosed subject matter, a method of delivering a therapeutic agent to a pregnant subject is provided. In some embodiments, an exemplary method includes administering to the pregnant subject an effective amount of a composition comprising ELPs disclosed herein coupled to one or more therapeutic agents. In some embodiments, ELPs with larger molecular weights accumulate in higher amounts in the placenta and/or are cleared slower from the plasma. In some embodiments, the method reduces the amount of the therapeutic agent crossing the placenta in a pregnant subject. Non-limiting examples of ELP which may be used in accordance with the presently-disclosed subject matter include ELPs having: about 32 repeats of SEQ ID NO: 1, where X is Val, Ala, and Gly in a 1:8:7 ratio (e.g., SEQ ID NO: 2); about 80 repeats of SEQ ID NO: 1, where X is Val, Ala, and Gly in a 1:8:7 ratio (e.g., SEQ ID NO: 3); about 160 repeats of SEQ ID NO: 1, where X is Val, Ala, and Gly in a 1:8:7 ratio (e.g., SEQ ID NO: 4); about 40 repeats of SEQ ID NO: 1, where X is Gly (e.g., SEQ ID NO: 5); about 80 repeats of SEQ ID NO: 1, where X is Gly (e.g., SEQ ID NO: 6); about 160 repeats of SEQ ID NO: 1, where X is Gly (e.g., SEQ ID NO: 7); about 32 repeats of SEQ ID NO: 1, where X is Val, Ala, or Gly in a 1:4:3 ratio (e.g., SEQ ID NO: 8); about 80 repeats of SEQ ID NO: 1 where X is Val, Ala, or Gly in a 1:4:3 ratio (e.g., SEQ ID NO: 9); about 160 repeats of SEQ ID NO: 1, where X is Val, Ala, or Gly in a 1:4:3 ratio (e.g., SEQ ID NO: 10); about 40 repeats of SEQ ID NO: 1, where X is Lys (e.g., SEQ ID NO: 11); about 80 repeats of SEQ ID NO: 1, where X is Lys (e.g., SEQ ID NO: 12); and about 160 repeats of SEQ ID NO: 1, where X is Lys (e.g., SEQ ID NO: 13). In some embodiments, the ELP sequence has an amino acid sequence selected from SEQ ID NOS: 2-13.

Additionally or alternatively, in some embodiments, the ELP is targeted to a specific region of the placenta. More specifically, the present inventors have surprisingly and unexpectedly found that specific sized ELP constructs target only particular regions of the placenta, while other sized ELP constructs simultaneously target multiple regions of the placenta. For example, an ELP composition having an ELP protein with a molecular weight of about 30 kDa or less, encoded by DNA containing about 70 or less repeat units of SEQ ID NO: 1, accumulates solely in the chorionic plate region of the placenta following administration. In contrast, an ELP composition having an ELP protein with a molecular weight of 47 kDa or greater, encoded by DNA containing greater than 120 repeat units of SEQ ID NO: 1, accumulates in the chorionic plate and labyrinth and junctional zone regions of the placenta following administration. Further increasing the size of the ELP to more than 200 repeat units of SEQ ID NO: 1, for example, increases the accumulation in labyrinth and junctional zone regions as compared to the ELP with 120 repeat units.

Accordingly, also provided herein are specifically targeted ELPs and methods of use thereof. In some embodiments, the targeted ELP is a chorionic plate targeting ELP. In some embodiments, the chorionic plate targeting ELP has a molecular weight of up to 30 kDa. For example, in one embodiment, the chorionic plate targeting ELP has a molecular weight of up to about 30 kDa, between 3 and about 30 kDa, or any combination, sub-combination, range, or sub-range thereof. In some embodiments, the chorionic plate targeting ELP includes up to 70 repeat units of SEQ ID NO: 1. For example, in one embodiment, the chorionic plate targeting ELP includes up to 70 repeat units, up to 63 repeat units, between 5 and 70 repeat units, between 31 and 63 repeat units of SEQ ID NO: 1, or any combination, sub-combination, range, or sub-range thereof.

In other embodiments, the targeted ELP is a chorionic plate and labyrinth/junctional zones targeting ELP. In some embodiments, the chorionic plate and labyrinth/junctional zones targeting ELP has a molecular weight of at least 40 kDa. For example, in one embodiment, the chorionic plate and labyrinth/junctional zones targeting ELP has a molecular weight of at least about 37 kDa, between about 50 and about 257 kDa, or any combination, sub-combination, range, or sub-range thereof. In some embodiments, the chorionic plate and labyrinth/junctional zones targeting ELP includes at least 95 repeat units of SEQ ID NO: 1. For example, in one embodiment, the chorionic plate and labyrinth/junctional zones targeting ELP includes at least about 95 repeat units, at least about 127 repeat units, between 127 and 671 repeat units of SEQ ID NO: 1, or any combination, sub-combination, range, or sub-range thereof.

Still further provided herein, in some embodiments, is a method of treating a disease or disorder in a pregnant subject, the method including administering one or more of the targeted ELPs to a subject in need thereof. In some embodiments, the method includes administering one or more of the chorionic plate targeting ELPs to a subject in need thereof. In some embodiments, the method includes administering one or more of the chorionic plate and labyrinth/junctional zones targeting ELPs to a subject in need thereof. In some embodiments, the method includes administering one or more of the primarily labyrinth/junctional zones targeting ELPs to a subject in need thereof. In some embodiments, the method includes administering a combination of one or more of the targeted ELPs disclosed herein. As will be appreciated by those skilled in the art, the selection of which targeted ELP will depend upon the specific disease or disorder being treated, as well as the therapeutic agent being used.

Turning now to the therapeutic agents that can be coupled to an exemplary ELP, various therapeutic agents known to those skilled in the art can be used in accordance with the presently-disclosed subject matter. As used herein, the terms “therapeutic agent” and “therapeutic agents” refer to any agent(s) that can be used in the “treatment” of a disease or disorder as defined herein below. In some embodiments, the therapeutic agent is selected from peptides, proteins, nucleic acids, antibodies, and small molecule drugs, or functional analogs thereof.

The terms “polypeptide,” “protein,” and “peptide,” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

As used herein, the term “analog” refers to any member of a series of peptides having a common biological activity, including antigenicity/immunogenicity and antiangiogenic activity, and/or structural domain and having sufficient amino acid identity as defined herein.

As noted, in certain embodiments of the presently-disclosed subject matter, the therapeutic agents coupled to ELPs are those therapeutic agents that are desirable for introduction into the maternal circulation, but that should preferably be prevented from crossing the placenta and entering fetal circulation. Thus, in some embodiments, the compositions described herein are useful for delivery of any type of therapeutic agent that is regarded as harmful to fetal development. Such therapeutic agents include, but are not limited to, agents for the treatment of preeclampsia, chemotherapeutics, many drugs for cardiovascular diseases, anti-epileptic drugs, anti-emetic drugs, many immune modulating agents for autoimmune disorders, many drugs for endocrine disorders, certain antibiotics and antivirals, some anti-inflammatory agents, hormonal agents, and some analgesics. A partial list of drugs in pregnancy category X (i.e., drugs with known fetal toxicities) are listed in Table 2 below. In addition, many other drugs in pregnancy categories C or D, which are identified as having some risk in pregnancy can benefit from delivery by coupling the drugs to an exemplary ELP in accordance with the presently-disclosed subject matter.

TABLE 2
Exemplary cytotoxic drugs capable of being coupled to an ELP,
including a partial list of Pregnancy Category X drugs (adapted
from Monthly Prescribing Reference, Jan. 9, 2013).
ALLERGIC DISORDERS
Vistaril (hydroxyzine) Early pregnancy
CARDIOVASCULAR SYSTEM

| | |
|---|---|
| Advicor (niacin ext-rel/lovastatin) | Letairis (ambrisentan) |
| Aggrenox (dipyridamole/aspirin) 3rd | Lipitor (atorvastatin) |
| trimester | Livalo (pitavastatin) |
| Altoprev (lovastatin) | Mevacor (lovastatin) |
| Bayer (aspirin) 3rd trimester | Multaq (dronedarone) |
| Caduet (amlodipine/atorvastatin) | Pravachol (pravastatin) |
| Coumadin (warfarin sodium) | Simcor (niacin ext-rel/simvastatin) |
| Crestor (rosuvastatin) | Tracker (bosentan) |
| Ecotrin (aspirin) 3rd trimester | Vytorin (ezetimibe/simvastatin) |
| Juvisync (sitagliptin/simvastatin) | Zocor (simvastatin) |
| Lescol (fluvastatin) | |
| Lescol XL (fluvastatin) | |

CENTRAL NERVOUS SYSTEM

| | |
|---|---|
| Beyaz (drospirenone/ethinyl estradiol) | Halcion (triazolam) |
| Doral (quazepam) | Restoril (temazepam) |
| Estazolam | Vistaril (hydroxyzine) Early pregnancy |
| Flurazepam | Yaz (drospirenone/ethinyl estradiol) |

DERMATOLOGICAL DISORDERS

| | |
|---|---|
| Amnesteem (isotretinoin) | Solaraze (diclofenac sodium) 3rd trimester |
| Avage (tazarotene) | Soriatane (acitretin) |
| Beyaz (drospirenone/ethinyl estradiol) | Sotret (isotretinoin) |
| Carac (fluorouracil) | SSD (silver sulfadiazine) Late pregnancy |
| Claravis (isotretinoin) | SSD AF (silver sulfadiazine) Late |
| Efudex (fluorouracil) | pregnancy |
| Estrostep Fe | Tazorac (tazarotene) |
| (norethindrone acetate/ethinyl estradiol) | Tilia Fe (norethindrone acetate/ethinyl |
| Fluoroplex (fluorouracil) | estradiol) |
| Loryna (drospirenone/ethinyl estradiol) | Tri-Legest 21 |
| Ortho Tri-Cyclen 28 | (norethindrone acetate/ethinyl estradiol) |
| (norgestimate/ethinyl estradiol) | Tri-Legest Fe |
| Propecia (finasteride) | (norethindrone acetate/ethinyl estradiol) |
| Silvadene (silver sulfadiazine) Late | Tri-previfem(norgestimate/ethinyl |
| pregnancy | estradiol) |
| Solage (mequinol/tretinoin) | Tri-sprintec (norgestimate/ethinyl |
| | estradiol) |
| | Yaz (drospirenone/ethinyl estradiol) |

ENDOCRINE DISORDERS

| | |
|---|---|
| Androderm (testosterone) | Lupron (leuprolide acetate) |
| Androgel (testosterone) | Methitest (methyltestosterone) |
| Android (methyltestosterone) | Oxandrin (oxandrolone) |
| Axiron (testosterone) | Striant (testosterone) |
| Delatestryl (testosterone enanthate) | Supprelin LA (histrelin acetate) |
| Depo-testosterone (testosterone | Synarel (nafarelin) |
| cypionate) | Testim (testosterone) |
| Egrifta (tesamorelin) | Testred (methyltestosterone) |
| Fluoxymesterone | Virilon (methyltestosterone) |
| Fortesta (testosterone) | |
| Juvisync (sitagliptin/simvastatin) | |

GASTROINTESTINAL TRACT

| | |
|---|---|
| Bellergal-S (phenobarbital/ergotamine | Cytotec (misoprostol) |
| tartrate) | |

INFECTIONS & INFESTATIONS

| | |
|---|---|
| Bactrim (sulfamethoxazole/trimethoprim) | Macrodantin |
| 3rd trimester | (nitrofurantoin macrocrystals) Pregnancy |
| Copegus (ribavirin) | at term |
| Flagyl (metronidazole) | Rebetol (ribavirin) |
| 1st trimester for trichomoniasis | Rebetron (ribavirin/interferon alfa -2b) |
| Furadantin (nitrofurantoin) Pregnancy at | Septra |
| term | (sulfamethoxazole/trimethoprim) 3rd |
| Gantrisin (sulfisoxazole) 3rd trimester | trimester |
| Grifulvin V (griseofulvin) | Sulfadiazine Pregnancy at term |
| Gris-Peg (griseofulvin) | Tindamax (tinidazole) 1st trimester |
| Macrobid (nitrofurantoin as | Urobiotic-250 |
| macrocrystals and monohydrate) Pregnancy | (oxytetracycline |
| at term | HCl/sulfamethizole/phenazopyridine) |
| | Late pregnancy |
| | Virazole (ribavirin) |

METABOLIC DISORDERS
Zavesca (miglustat)
MUSCULOSKELETAL DISORDERS

| | |
|---|---|
| Advil (ibuprofen) 3rd trimester | Evista (raloxifene HCl) |
| Aleve (naproxen sodium) 3rd trimester | Feldene (piroxicam) Late pregnancy |
| Ansaid (flurbiprofen) Late pregnancy | Ibuprofen 3rd trimester |
| Arava (leflunomide) | Ketoprofen Late pregnancy |
| Arthrotec (diclofenac sodium/ | Mobic (meloxicam) 3rd trimester |
| misoprostol) | Nabumetone 3rd trimester |
| Bayer (aspirin) 3rd trimester | Nalfon (fenoprofen calcium) 3rd trimester |
| BC Arthritis Strength | Naprelan (naproxen) 3rd trimester |
| (aspirin/caffeine/salicylamide) 3rd | Prevacid Naprapac |
| trimester | (lansoprazole/naproxen) 3rd trimester |
| Cataflam (diclofenac potassium) Late | Probenecid + Colchicine |
| pregnancy | Prolia (denosumab) |
| Celebrex (celecoxib) 3rd trimester | Rheumatrex (methotrexate sodium) |
| Choline magnesium trisalicylate | Salsalate 3rd trimester |
| Pregnancy at term | Soma Compound w. Codeine |
| Dantrium (dantrolene) | (carisoprodol/aspirin/codeine) 3rd |
| Daypro (oxaprozin) 3rd trimester | trimester |
| Diclofenac sodium Late pregnancy | Vimovo (naproxen/esomeprazole) Late |
| Difluni sal 3rd trimester | pregnancy (>30 wks) |
| Duexis (ibuprofen/famotidine) | Zipsor (diclofenac potassium) Late |
| Late pregnancy (>30 wks) | pregnancy |
| Ecotrin (aspirin) 3rd trimester | |
| Etodolac Late pregnancy | |

NEOPLASMS

| | |
|---|---|
| Bexxar (tositumomab) | Menest (esterified estrogens) |
| Casodex (bicalutamide) | Revlimid (lenalidomide) |
| Delestrogen (estradiol valerate) | Targretin (bexarotene) |
| Efudex (fluorouracil) | Thalomid (thalidomide) |
| Eligard (leuprolide acetate) | Trelstar (triptorelan pamoate) |
| Estrace (estradiol) | Trexall (methotrexate) |
| Evista (raloxifene HCl) | Vantas (histrelin acetate) |
| Firmagon (degarelix) | Zytiga (abiraterone acetate) |
| Fluoxymesterone | |

NUTRITION

| | |
|---|---|
| Didrex (benzphetamine) | Megace Suspension (megestrol acetate) |
| Fosteum (genistein/citrated | Xenical (orlistat) |
| zinc/cholecalciferol) | |
| Megace ES (megestrol acetate) | |

OB/GYN

| | |
|---|---|
| ALL ORAL CONTRACEPTIVES | Lupron Depot (leuprolide acetate) |
| ALL HORMONE REPLACEMENT | Luveris (lutropin alfa) |
| THERAPY | Menopur (menoptropins) |
| Advil (ibuprofen) 3rd trimester | Methergine (methylergonovine) |
| Aleve (naproxen sodium) 3rd trimester | Midol cramp (ibuprofen) 3rd trimester |
| Aygestin (norethindrone acetate) | Midol menstrual |
| Betadine douche (povidone-iodine) | (acetaminophen/caffeine/pyrilamine) 3rd |
| Bravelle (urofollitropin) | trimester |
| Cataflam (diclofenac potassium) Late | Midol PMS |
| pregnancy | (acetaminophen/pamabrom/pyrilamine) |
| Celebrex (celecoxib) 3rd trimester | 3rd trimester |
| Cetrotide (cetrorelix) | Midol teen (acetaminophen/pamabrom) |
| Clomid (clomiphene citrate) | 3rd trimester |
| Depo-subQ provera | Mifeprex (mifepristone) |
| (medroxyprogesterone acetate) | Naprelan (naproxen) 3rd trimester |
| Endometrin (micronized progesterone) | Ovidrel (choriogonadotropin alfa) |
| Ectopic pregnancy | Ponstel (mefenamic acid) Late pregnancy |
| Flagyl (metronidazole) | Repronex 75 IU |
| 1st trimester for trichomoniasis | (follicle-stimulating hormone/luteinizing |
| Follistim (follitropin beta) | hormone) |
| Ganirelix acetate | Repronex 150 IU |
| Gonal-F (follitropin alfa) | (follicle-stimulating hormone/luteinizing |
| Ibuprofen 3rd trimester | hormone) |
| | Serophene (clomiphene citrate) |
| | Synarel (nafarelin acetate) |
| | Tindamax (tinidazole) 1st trimester |
| | Zoladex (goserelin) |

PAIN MANAGEMENT

| | |
|---|---|
| Advil (ibuprofen) 3rd trimester | Excedrin Migraine |
| Advil Migraine (ibuprofen) 3rd trimester | (acetaminophen/aspirin/caffeine) 3rd |
| Aleve (naproxen sodium) 3rd trimester | trimester |
| Bayer (aspirin) 3rd trimester | Fiorinal (butalbital/aspirin/caffeine) 3rd |
| BC Original Formula | trimester |
| (aspirin/caffeine/salicylamide) 3rd | Fiorinal w. Codeine |
| trimester | (butalbital/aspirin/caffeine/codeine |
| Cafergot (ergotamine tartrate/ caffeine) | phosphate) 3rd trimester |
| Cal dolor (ibuprofen) 3rd trimester | Ibudone |
| Cataflam (diclofenac potassium) Late | (hydrocodone bitartrate/ibuprofen) 3rd |
| pregnancy | trimester |
| Celebrex (celecoxib) 3rd trimester | Ketorolac Late pregnancy |
| Choline magnesium trisalicylate | Migranal (dihydroergotamine mesylate) |
| Pregnancy at term | Motrin Migraine Pain (ibuprofen) 3rd |
| D.H.E. 45 (dihydroergotamine mesylate) | trimester |
| Diflunisal 3rd trimester | Nalfon (fenoprofen calcium) 3rd trimester |
| Etodolac Late pregnancy | Naprelan (naproxen) 3rd trimester |
| | Percodan (oxycodone HCl/aspirin) 3rd |
| | trimester |
| | Ponstel (mefenamic acid) Late pregnancy |
| | Synalgos-DC |
| | (dihydrocodeine bitartrate/aspirin/caffeine) |
| | 3rd trimester |
| | Vicoprofen (hydrocodone |
| | bitartrate/ibuprofen) 3rd trimester |

UROGENITAL SYSTEM

| | |
|---|---|
| Avodart (dutasteride HCl) AVOID | Jalyn (dutasteride/tamsulosin HCl) |
| HANDLING CAPSULES | Lithostat (acetohydroxamic acid) |
| Caverject (alprostadil) | Proscar (finasteride) |
| Edex (alprostadil) | |

Additionally, the presently-disclosed subject matter is not limited to delivery of small molecule drugs, but is also useful for delivery of peptide agents, therapeutic proteins, and antibodies. A partial list of such other types of agents that can be improved by ELP delivery during pregnancy is included in Table 3 below.

TABLE 3
Partial list of peptide, protein, and antibody agents that
can be coupled to a ELP for delivery during pregnancy.

| | | | |
|---|---|---|---|
| | | GenBank No. | |
| | Protein of | (of gene from which | |
| Peptide Name | origin | peptide is derived) | Amino Acids |

THERAPEUTIC PEPTIDES

| | | | |
|---|---|---|---|
| PNC-2 | Ras | 3265 | 96-110 |
| PNC-7 | Ras | 3265 | 35-47 |
| PNC-25 | SOS | 6654 | 994-1004 |
| n.s.* | Raf | 5894 | 97-110 |
| n.s.* | Raf | 5894 | 143-150 |
| n.s.* | NF1-GAP | 4763 | 1121-1128 |
| SP1068 | EGFR | 1956 | 1063-1073 |
| SY317 | Shc | 6464 | 312-323 |
| n.s.* | MEK1 | 5604 | 1-13 |
| n.s.* | GST-pi | 2950 | 34-50 |
| JNKI1 | JIP1/IB1 | 9479 | 153-172 |
| JNKI2 | JIP2/IB2 | 9479 | 134-151 |
| I-JIP | JIP1/IB1 | 9479 | 143-163 |
| TI-JIP | JIP1/IB1 | 9479 | 153-163 |
| NBD | IKKβ | 3551 | 735-745 |
| CC2 | NEMO | 8517 | 253-287 |
| LZ | NEMO | 8517 | 294-336 |
| SN50 | NF-κB p50 | 4790 | 360-369 |
| pp21 | IκBα | 4792 | 21-41 |
| ρ65-P1 | NF-κB p65 | 5970 | 271-282 |
| p65-P6 | NF-κB p65 | 5970 | 525-537 |
| C1 | p53 | 7157 | 369-383 |
| Peptide 46 | p53 | 7157 | 361-382 |
| CDB3 | 53BP2 | 7159 | 490-498 |
| TIP | p53 | 7157 | 12-30 |
| Super-TIP | (phage selected) | | |
| PNC-27 | p53 | 7157 | 12-26 |
| PNC-21 | p53 | 7157 | 12-20 |
| PNC-28 | p53 | 7157 | 17-26 |
| αHDM2 | p53 | 7157 | 16-27 |
| Peptide 3 | p14^{ARF} | 1029 | 1-20 |
| H1-S6A, F8A | c-Myc | 4609 | 368-381 |
| n.s.* | p21 | 1026 | 17-33 |
| n.s.* | p21 | 1026 | 63-77 |
| Peptide 10 | p21 | 1026 | 141-160 |
| W10 | p21 | 1026 | 139-164 |
| Peptide 6 | p16 | 1029 | 84-103 |
| Peptide 5a | p27 | 1027 | Modified |
| | | | from 30-34 |
| C4 | cyclin A | 890/8900 | 285-306 |
| n.s.* | E2F | 1869 | 87-64 |
| n.s.* | Rb | 5925 | 864-880 |
| Akt-in | TCL1 | 8115 | 10-24 |
| Peptide2 | FKHRL1 | 2309 | 16-24 |
| n.s.* | Bak | 578 | 72-87 |
| TO4 | Bax | 581 | 52-72 |
| n.s.* | Bax | 581 | 53-86 |
| n.s.* | Bad (mus | 12015 | 140-165 |
| | musculis) | | |
| n.s.* | Bad | 572 | 103-127 |
| BH3 BAD | Bad | 572 | 103-123 |
| Bim | Bim | 10018 | 145-165 |
| n.s.* | Bid | 637 | 84-99 |
| SAHB_{A} | Bid | 637 | 80-101 |
| Smac-7 | Mature Smac | 56616 | 1-7 |
| n.s.* | Mature Smac | 56616 | 1-4 |
| dAVPI | Mature Smac | 56616 | 1-4 |
| Nox2ds | NADPH | 1536 | 86-94 |
| | oxidase 2 | | |
| Nox2 C-terminal peptide 1 | NADPH | 1536 | 552-570 |
| | oxidase 2 | | |
| Nox2 C-terminal peptide 2 | NADPH | 1536 | 550-569 |
| | oxidase 2 | | |
| Nox2 C-terminal peptide | NADPH | 1536 | 491-504 |
| (with mutation at residue 500) | oxidase 2 | | |
| p22^{phox} derived peptide 1 | p22^{phox} | 1535 | 9-23 |
| p22^{phox} derived peptide 2 | p22^{phox} | 1535 | 31-45 |
| p22^{phox} derived peptide 3 | p22^{phox} | 1535 | 47-61 |
| p22^{phox} derived peptide 4 | p22^{phox} | 1535 | 85-99 |
| p22^{phox} derived peptide 5 | p22^{phox} | 1535 | 113-127 |
| p22^{phox} derived peptide 6 | p22^{phox} | 1535 | 82-95 |
| p22^{phox} derived peptide 7 | p22^{phox} | 1535 | 175-194 |
| p47^{phox} derived peptide 1 | p47^{phox} | 653361 | 323-332 |
| p47^{phox} derived peptide 2 | p47^{phox} | 653361 | 314-331 |
| p47^{phox} derived peptide 3 | p47^{phox} | 653361 | 315-328 |
| p47^{phox} derived peptide 4 | p47^{phox} | 653361 | 323-332 |
| p47^{phox} derived peptide 5 | p47^{phox} | 653361 | 334-347 |
| * n.s., name not specified | | | |

THERAPEUTIC PROTEINS

| | | |
|---|---|---|
| VEGF | Insulin | β-Gluco- |
| | | cerebrosidase |
| PlGF | Growth hormone | Alglucosidase-α |
| IL10 | Mecasermin | Laronidase |
| IL11 | Factor VIII | Idursulphase |
| Erythropoietin | Factor IX | Galsulphase |
| Darbepoetin | Antithormbin III | Agalsidase-β |
| G-CSF | Protein C | α-1-Proteinase |
| | | inhibitor |
| Peg-G-CSF | tPA | Lipase |
| GM-CSF | Urokinase | Amylase |
| α-interferon | Factor VIIa | Adenosine |
| | | deaminase |
| Interferon-α2a | Calcitonin | Albumin |
| Interferon-α2b | Teriparatide | FSH |
| Peg-Interferon-α2a | Exenatide | HCG |
| Peg-Interferon-α2b | Octreotide | Lutropin |
| Interferon-αN3 | rhBMP2 | Nesiritide |
| Interferon-β1a | rhBMP7 | Botulinum Toxin |
| | | type A |
| Interferon-β1b | GnRH | Botulinum Toxin |
| | | type B |
| Interferon-γ1b | KGF | Collagenase |
| IL2 | PDGF | DNAse I |
| ETAF | Trypsin | Hyaluronidase |
| Peg-Asparaginase | Bivalirudin | Papain |
| Rasbuicase | Streptokinase | L-Asparaginase |
| Lepirudin | Anistreplase | |

ANTIBODIES

| | | |
|---|---|---|
| Bevacizumab | Abatacept | Basiliximab |
| Cetuximab | Anakinra | Daclizumab |
| Panitumumab | Adalimumab | Muromonab-CD3 |
| Alemtuzumab | Etanercept | Omalizumab |
| Rituximab | Infliximab | Palivizimuab |
| Trastuzumab | Alefacept | Enfuvirtide |
| Ranibuzumab | Efalizumab | Abciximab |
| Denileukin diftitox | Natalizumab | Pegvisomant |
| Ibritumomab tiuxetan | Eculizumab | GHRH |
| Gemtuzumab ozogamicin | DPPD | Secretin |
| Tositumomab | Glucagon | TSH |
| Capromab pendetide | Indium-111-ocreotide | Satumomab |
| | | pendetide |
| Arcitumomab | Nofetumomab | Apcitide |
| Imciromab pentetate | Technetium fanolesomab | |

In some embodiments, the therapeutic agent coupled to the ELP and used in accordance with the presently-disclosed subject matter is a therapeutic agent useful for the treatment of preeclampsia, eclampsia, myocardial infarction, renovascular disease, spinocerebellar ataxia, lupus, rheumatoid arthritis, psoriatic arthritis, multiple sclerosis, cancer, Crohn's disease, ankylosing spondylitis, cardiac hypertrophy, plaque psoriasis, hypertension, atherosclerosis, stroke, kidney stones, Alzheimer's disease and other neurodegenerative disorders, prevention of allograft rejection, hepatic fibrosis, schizophrenia, muscular dystrophy, macular degeneration, pulmonary edema, chronic pulmonary hypertension, or other disorders where ROS are deleterious. In some embodiments, the therapeutic agent coupled to the ELP is selected from the therapeutic agents listed in Tables 2 and 3.

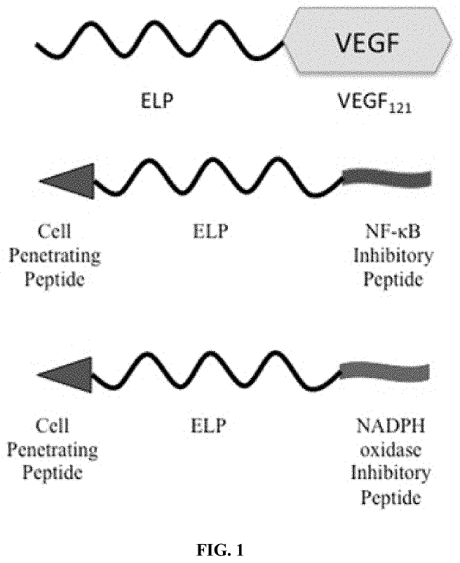

In certain embodiments, the therapeutic agent coupled to the ELP is an isoform of vascular endothelial growth factor (VEGF) (FIG. 1). As would be recognized by those skilled in the art, VEGF is a signaling protein that plays a potent role in vasculogenesis and angiogenesis. Many diseases are associated with decreased VEGF levels or increases in antiangiogenic proteins that antagonize VEGF such as sFlt-1. For example, in preeclampsia, the ischemic placenta releases large amounts of sFlt-1 into the maternal circulation that antagonizes VEGF effects both in the placenta and throughout the maternal vasculature (Clark D E, et al., 1998). VEGF levels are reduced in other diseases as well, and VEGF supplementation has been shown to be beneficial for treatment of myocardial infarction, renovascular disease, and spinocerebellar ataxia (Banai S, et al., 1994; Pearlman J D, et al., 1995; Chade A R, et al., 2012; Chade A R, 2012; Cvetanovic M, et al., 2011). However, the therapeutic strategy used to treat these diseases is not as straightforward as simply infusing VEGF. Free exogenous VEGF is very short-lived, with a plasma half-life in humans of about 34 minutes (as determined following a four hour intravenous infusion of recombinant human VEGF₁₆₅) (Eppler S M, et al., 2002). Due to the short half-life and poor stability of the protein, constant infusion via a pump-driven catheter placed directly at the diseased site is required. This type of treatment strategy is not a viable translational approach for many diseases, where patients may need to be treated for long periods of time. The second limitation of free VEGF supplementation that is specific for preeclampsia therapy involves its potential for damage to the developing fetus. Several reports have demonstrated the severe potential consequences of overloading the fetus with VEGF. Overexpression of VEGF-A by two to three-fold using a genetic strategy in mouse embryos resulted in embryonic lethality at day E12.5 (Miquerol L, et al., 2000). A separate study in which quail embryos were directly injected with exogenous VEGF showed similar results (Drake C J, et al., 1995). In both studies, these VEGF treated embryos also had malformation of the hearts, including fusion of inflow and aortic outflow channels. These studies address the dire consequences of increasing VEGF levels directly in the developing fetus, but it has also been shown that administration of free VEGF to pregnant mice causes developmental problems in the embryos. Daily systemic injection of recombinant human VEGF from gestational day 9 to day 17 resulted in an 18-fold increase in the fetal resorption rate and a significant decrease in fetal weight among the surviving fetuses (He Y, et al., 1999). Given the limitations of short half-life and the potential for teratogenic effects of free VEGF, and without wishing to be bound by any particular theory, it is believed that, by fusing VEGF to the ELP carrier, VEGF's plasma half-life can be extended while preventing its delivery across the placenta. In some embodiments, the composition comprises a ELP coupled to a VEGF sequence. One non-limiting example of the ELP-VEGF sequences is a ELP sequence (SEQ ID NO: 4) fused to a C-terminal VEGF₁₂₁ sequence (SEQ ID NO: 14).

In yet further embodiments, the therapeutic agent is an NF-κB inhibitory peptide (FIG. 1). As would also be recognized by those in the art, inflammation is a hallmark of many diseases, including preeclampsia. A theme in all of these inflammatory processes is the production of pro-inflammatory cytokines, such as interleukins, INF-γ, and TNF-α. In this regard, many pro-inflammatory cytokines such as TNF-α exert their effects via receptor-mediated signaling pathways that are centrally routed through NF-κB. NF-κB activation upon extracellular signaling is mediated by phosphorylation and release of the natural inhibitor I-κB from the NF-κB p50/p65 heterodimer. I-κB release exposes a nuclear localization sequence (NLS) on the p50 subunit of NF-κB, and once exposed, this NLS mediates nuclear import of NF-κB. Once inside the nucleus, NF-κB then binds to response elements on its target genes and regulates gene expression. In this regard, in some embodiments, a synthetic cell permeable peptide containing a p50 NLS capable of blocking the nuclear import of NF-κB upon stimulation in a variety of cell lines is fused to an exemplary ELP carrier described herein. In some embodiments, and as described in further detail below, such a polypeptide also contains a cell penetrating peptide (CPP) to mediate uptake into target cells. A non-limiting example of the composition is shown as SynB1-ELP-p50 (SEQ ID NO: 15) where a SynB1 peptide fused to N-terminus of a ELP sequence (SEQ ID NO: 4), and a p50 peptide sequence fused to the C-terminus of the ELP sequence.

In still other embodiments, the therapeutic agent coupled to the ELP is a NADPH oxidase inhibitory peptide (FIG. 1). Another contributing factor to many cardiovascular disorders, including preeclampsia, is the production of reactive oxygen species (ROS). ROS are a natural byproduct of metabolism, but if produced in excessive levels, they can cause damage to key cellular components. For example, high ROS levels can induce DNA damage, lipid peroxidation in the plasma membrane, and oxidation of cellular proteins, and downstream results of these effects can include cell death. One major producer of the ROS superoxide is NADPH oxidase (NOX). NOX activity has been shown to be important for pathological ROS production in hypertension, atherosclerosis, stroke, preeclampsia, kidney stones, Alzheimer's disease and other neurodegenerative disorders, schizophrenia, muscular dystrophy, macular degeneration, pulmonary edema, chronic pulmonary hypertension, among others (Paravicine T M, et al., 2008; Park Y M, et al., 2009; Radermacher K A, et al., 2013; Matsubara S, et al., 2001; Khan S R, 2013; Block M L, 2008; Wang X, et al., 2013; Whitehead N P, et al., 2010; Monaghan-Benson E, et al., 2010; Araneda O F, et al., 2012). Thus, in some embodiments, a peptide inhibitor of NADPH oxidase called Nox2ds (abbreviated NOX) is coupled to an ELP. NOX is a 9 amino acid sequence from the cytosolic portion of Nox2 that prevents the interaction of the p47phox structural subunit with Nox2 (Cifuentes-Pagano E, et al., 2012; Csanyi G, et al., 2011). In some embodiments, a CPP is also coupled to the NOX polypeptide composition to mediate its uptake into target cells. A non-limiting sequence is shown as SynB1-ELP-NOX (SEQ ID NO: 16) where SynB1 peptide sequence fused to the N-terminus of a ELP sequence (SEQ ID NO: 4), and NOX peptide fused to the C-terminus of the ELP sequence.

In some embodiments, the therapeutic agent is a small molecule drug, where the size of the small molecule drug is less than 2,000 Dalton. In some embodiments, the small molecule drug is known to cause adverse events during pregnancy. Non-limiting examples of adverse events include teratogenicity, fetal growth restriction, embryotoxicity, or fetal demise. In some embodiments, the small molecules include pregnancy category C, D, or X drugs classified by the US FDA (Federal Register, Vol. 73, No. 104, May 29, 2008; Postmarket Drug Safety Information for Patients and Providers, Index to Drug-Specific Information). In some embodiments, the small molecule drug includes anti-hypertensive drugs. Non-limiting examples of the anti-hypertensive drugs include lovastatin, atorvastatin, pitavastatin, pravastatin, simvastatin, rosuvastatin, fluvastatin, aspirin, captopril, zofenopril, enalapril, ramipril, perindopril, quinapril, lisinopril, cilazapril, trandolapril, benazepril, imidapril, foninopril. In some embodiments, the small molecule drug includes anti-epileptic agents. Non-limiting examples of the anti-epileptic agents include phenytoin, valproate, phenobarbital, valproic acid, trimethadione, paramethadione, topiramate, carbamazepine, levetiracetam, lamotrigine. In some embodiments, the small molecule drug includes anti-emetic drugs. Non-limiting anti-emetic drugs include doxylamine, pyridoxine, prochlorperazine, chlorpromazine, promethazine, trimethobenzamide, ondansetron. In some embodiments, the small molecule drug includes cancer chemotherapeutics. Non-limiting examples of the cancer chemotherapeutics are taxanes including paclitaxel and decetaxel; vinca alkyloids including vinblastine, vincristine, venorelbine, and vinflunin; antimetabolites including methotrexate and 5-fluorouracil; topoisomerase inhibitors including doxorubicin, daunorubicin, epirubicin, etoposide, and camptothecin; cyclophosphamide or related alkylating agents.

Various means of coupling the ELP to therapeutic agents can be used in accordance with the presently-disclosed subject matter and are generally known to those of ordinary skill in the art. Such coupling techniques include, but are not limited to, chemical coupling and recombinant fusion technology. Depending on the particular coupling techniques utilized, in some embodiments, the number of ELPs or therapeutic agents per molecule, and their respective positions within the molecule, can be varied as needed. Further, in some embodiments, the therapeutic agent may further include one or more spacer or linker moieties, which in addition to providing the desired functional independence of the ELP and therapeutic agents, can optionally provide for additional functionalities, such as a protease-sensitive feature to allow for proteolytic release or activation of the therapeutic agent. Moreover, in certain embodiments, the therapeutic agent may be coupled to one or more targeting components such as, for example, a peptide or protein that targets the therapeutic agent to a particular cell type, e.g., a cancer cell, or to a particular organ, e.g., the liver.

To facilitate entry of the peptide compositions described herein into a cell where the therapeutic effect of the compositions can be exerted, in some embodiments, the polypeptide compositions further include a cell-penetrating peptide (CPP) sequence or an organ targeting peptide sequence that is coupled to the ELP.

As used herein, the term “cell penetrating peptide” refers to peptides sequences that facilitate cellular uptake of various agents, such as polypeptides, nanoparticles, small chemical molecules, and fragments of DNA. The function of the CPPs are to deliver the agents into cells, a process that commonly occurs through endocytosis with the cargo delivered to the endosomes of living mammalian cells. In some embodiments, the cell penetrating peptides or organ targeting peptides couple to the ELP carrier either through chemical linkage via covalent bonds or through non-covalent interactions. Non-limiting examples of the cell-penetrating peptide that can be coupled to the therapeutic agent or ELP include penetratin, Tat, SynB1, Bac, polyArg, MTS, Transportan, or pVEC.

The term “organ targeting peptide” refers to peptides designed to have specificity for the vascular beds or other cell types of specific organs. In some embodiments, the organ targeting peptide is selected from kidney targeting peptides, placenta targeting peptides, or brain targeting peptides.

Further provided, in some embodiments of the presently-disclosed subject matter are methods for the treatment of various diseases and disorders using the exemplary ELP-therapeutic agent-containing compositions described herein. In some embodiments, the presently-disclosed subject matter includes a method of treating a disease or disorder in a pregnant subject wherein the pregnant subject is administered an effective amount of a composition comprising an ELP coupled to a therapeutic agent, wherein the ELP is at least 5 repeats of SEQ ID NO: 1. Exemplary diseases or disorders that can be treated in accordance with the presently-disclosed subject matter include, but are not limited to, preeclampsia, eclampsia, myocardial infarction, renovascular disease, spinocerebellar ataxia, lupus, rheumatoid arthritis, psoriatic arthritis, multiple sclerosis, cancer, Crohn's disease, ankylosing spondylitis, cardiac hypertrophy, plaque psoriasis, hypertension, atherosclerosis, stroke, kidney stones, Alzheimer's disease and other neurodegenerative disorders, prevention of allograft rejection, hepatic fibrosis, schizophrenia, muscular dystrophy, macular degeneration, pulmonary edema, chronic pulmonary hypertension, or other disorders where ROS are deleterious.

As used herein, the terms “treatment” or “treating” relate to any treatment of a disease or disorder, including but not limited to prophylactic treatment and therapeutic treatment. As such, the terms “treatment” or “treating” include, but are not limited to: preventing a disease or disorder or the development of a disease or disorder; inhibiting the progression of a disease or disorder; arresting or preventing the further development of a disease or disorder; reducing the severity of a disease or disorder; ameliorating or relieving symptoms associated with a disease or disorder; and causing a regression of a disease or disorder or one or more of the symptoms associated with a disease or disorder.

For administration of a therapeutic composition as disclosed herein (e.g., an ELP coupled to a therapeutic agent), conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082).

Regardless of the route of administration, the compositions of the presently-disclosed subject matter are typically administered in an amount effective to achieve the desired response. As such, the term “effective amount” is used herein to refer to an amount of the therapeutic composition (e.g., en ELP coupled to a therapeutic agent, and a pharmaceutical vehicle, carrier, or excipient) sufficient to produce a measurable biological response. Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

In some embodiments of the presently-disclosed subject matter, the compositions described herein have been found to be particularly useful for the treatment of preeclampsia during pregnancy. However, it is contemplated that the exemplary compositions described are also useful not only for the treatment of a number of other diseases and disorders, but also both during pregnancy and in non-pregnant populations. For example, the ELP-delivered VEGF can be useful for treatment of myocardial infarction, renovascular disease, spinocerebellar ataxia, or other disorders in which VEGF levels are reduced. Additionally, the ELP-delivered NF-κB inhibitory peptide (SEQ ID NO: 17) could be useful for a variety of disorders with an inflammatory component, including lupus, rheumatoid arthritis, psoriatic arthritis, multiple sclerosis, cancer, Crohn's disease, ankylosing spondylitis, cardiac hypertrophy, plaque psoriasis, or other disorders in which NF-κB plays a central regulatory role. Moreover, the ELP-delivered NOX peptide could be used for hypertension, atherosclerosis, stroke, kidney stones, Alzheimer's disease and other neurodegenerative disorders, prevention of allograft rejection, hepatic fibrosis, schizophrenia, muscular dystrophy, macular degeneration, pulmonary edema, chronic pulmonary hypertension, or other disorders where ROS are deleterious.

In addition to the advantageous properties and uses described above, and without wishing to be bound by any particular theory, it is believed that the fusion of therapeutic agents to the ELP carrier provides many other advantages as well. For instance, in certain embodiments, ELP fusion increases the plasma half-life of therapeutic agents as many small molecule drugs, peptides, and therapeutic proteins are typically rapidly cleared from circulation by renal filtration. As another example, in some embodiments, ELP fusion increases the solubility of therapeutics as ELP fusion has been shown to increase the solubility of many poorly soluble therapeutics. As yet another example, in some embodiments, ELP fusion protects labile peptide therapeutics from degradation in vivo as ELP fusion provides a large sized carrier for labile therapeutics that protects them from enzymes that would degrade them (Bidwell G L, et al., 2013; Bidwell G L, 3rd, et al., 2012). Further, in some embodiments, ELP fusion decreases the immunogenicity of therapeutics that may be otherwise recognized as foreign by the immune system as ELP has been shown to be non-immunogenic and to decrease the immunogenicity of attached therapeutics.

As an additional example of the advantageous use of an ELP, in some embodiments, the ELP sequence can be easily modified to carry any desired protein or peptide, or to incorporate labeling sites for attachment of small molecules. Indeed, when an ELP is genetically encoded, and its coding sequence is inserted into a plasmid vector, doing so allows manipulation of the ELP sequence, and fusions of peptides and therapeutic proteins can be made by molecular biology techniques (Bidwell G L, 2012; Bidwell G L, et al., 2005; Bidwell G L, et al., 2010; Bidwell G L, 3rd, Wittom A A, et al., 2010; Massodi I, et al., 2005; Massodi I, et al., 2009; Meyer D E, 1999; Moktan S, et al., 2012; Moktan S, et al., 2012). Moreover, ELP can be purified after recombinant expression in bacteria. The genetically encoded nature of ELP also allows for expression in bacteria. Large amounts of ELP or ELP fusion proteins can be expressed recombinantly using E. coli-based expression systems. Additionally, ELP has the property of being thermally responsive. Above a characteristic transition temperature, ELP aggregates and precipitates, and when the temperature is lowered below the transition temperature, ELP re-dissolves. Therefore, purification of ELP after expression in bacteria can include heating the bacterial lysate above the transition temperature and collecting ELP or ELP fusion proteins by centrifugation. Repeated centrifugations above and below the transition temperature then results in pure ELP (Meyer D E, et al., 1999).

Furthermore, in some embodiments of the presently-disclosed subject matter, by using ELPs, ELPs can be targeted to desired tissues in vivo using targeting agents or peptides. As noted above, because of the ease of generating ELP fusions, ELP can be conjugated with any targeting agent, be it a peptide, small molecule, or antibody. Indeed, fusion with CPPs or organ targeting peptides can be used to not only increase cell and tissue uptake of ELPs, but also to direct ELP to specific tissues in vivo and even to specific intracellular compartments within a particular subject (Bidwell G L, et al., 2013; Bidwell G L, et al., 2009).

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Polynucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples.

### EXAMPLES

### Example 1. Design and Method for Generation of ELP-Fusion Polypeptides

ELP sequences were made by recursive directional ligation. A synthetic nucleotide cassette containing the coding sequence for 5 to 10 VPGXG (SEQ ID NO: 1) repeats with the desired amino acids at the X position and flanked by PflMI and BlgI restriction sites was cloned into the pUC19 vector at the EcoRI and HinDIII sites. The sequence of this construct was confirmed by DNA sequencing using standard M13 forward and reverse primers. Once one block of 5 to 10 VPGXG (SEQ ID NO: 1) repeats was inserted and confirmed, it was excised from pUC19 using PflMI and BglI restriction digestion and purified using agarose electrophoresis. A second aliquot of pUC19 containing the VPGXG (SEQ ID NO: 1) repeated sequence was linearized by digestion with PflMI only, and the gel purified cassette was ligated into the PflMI restriction site. This resulted in an in-frame fusion of the block of 5-10 VPGXG (SEQ ID NO: 1) repeats with a second block of 5 to 10 VPGXG (SEQ ID NO: 1) repeats, effectively doubling the number of ELP repeats. This process was repeated, doubling the ELP repeat number each time, until the desired molecular weight was reached. If necessary, smaller blocks (such as the original 5-10 repeat block) were used to increase the ELP repeat size in 5 to 10 block increments until the exact desired VPGXG (SEQ ID NO: 1) repeat number was achieved. The final ELP sequence was then excised from pUC19 using PflMI and BglI and inserted into a modified pET25b expression vector at an engineered SfiI site for recombinant protein expression.

N- and C-terminal modifications of ELP were made by cloning desired N- and/or C-terminal peptide or protein coding sequence into the pET25b expression vector between the NdeI and BamHI restriction sites. In all cases, the N- and/or C-terminal modifications were separated by an SfiI restriction site for later insertion of ELP. For peptide modification (such as CPPs, the NADPH oxidase inhibitory peptide (SEQ ID NO: 18), or the NF-κB inhibitory peptide (SEQ ID NO: 17)), the coding sequence for the peptides was generated as a synthetic oligonucleotide cassette with ends compatible with the desired restriction sites. For larger protein insertions, such as VEGF, the coding sequence was either commercially synthesized with E. coli-optimized codons and flanked by the desired restriction sites, or the coding sequence was amplified from human cDNA by PCR with custom primers used to add any necessary N- or C-terminal amino acids and to add the desired restriction sites. The intermediate constructs containing only the N- and/or C-terminal modifications in the pET25b vector were confirmed by DNA sequencing using the standard T7 promoter and T7 terminator primers. The desired ELP coding sequence was extracted from pUC19 using PflMI and BglI digestion and cloned into the modified pET25b vector at the engineered SfiI site. This resulted in in-frame fusions of ELP with the desired N- and/or C-terminal peptide or protein modifications. The final constructs were again confirmed by DNA sequencing.

### Example 2. Recombinant Expression and Purification of Polypeptides

ELPs and ELP fusion proteins were expressed and purified from E. coli BLR (DE3) or Rosetta2®(DE3) (for constructs resulting from human cDNA containing human-optimized codons). Briefly, 500 mL of TB Dry liquid culture media (MoBio) was inoculated with the expression strain and cultured at 37° C. with 250 rpm agitation for 16-18 h. In the absence of the pLysS lysozyme-expressing plasmid, the pET expression system allows for leaky production of the recombinant protein even without inducing agents. Bacteria were harvested by centrifugation and lysed by sonication (10×10 sec pulses, 75% amplitude, Fisher Sonic Dismembrator). Cell debris was removed by centrifugation, and nucleic acids were precipitated with 10% polyethylene imine and removed by centrifugation. NaCl was added to the soluble bacterial lysate to lower the ELP transition temperature (4 g/30 mL), and the lysate was heated to 42° C. to induce aggregation of the ELP-containing polypeptides. Polypeptides were collected by centrifugation at 42° C., the supernatant containing other soluble proteins was discarded, then the ELPs or ELP-fusion proteins were re-solubilized in ice cold PBS. Any remaining debris was removed after re-dissolving the ELP-containing proteins by centrifugation at 4° C. This heat-induced aggregation process was repeated two to three times to achieve purified ELP or ELP-fusion proteins. Purity of the resulting polypeptides was confirmed by SDS-PAGE analysis.

### Example 3. Use of ELP for Drug Delivery During Pregnancy

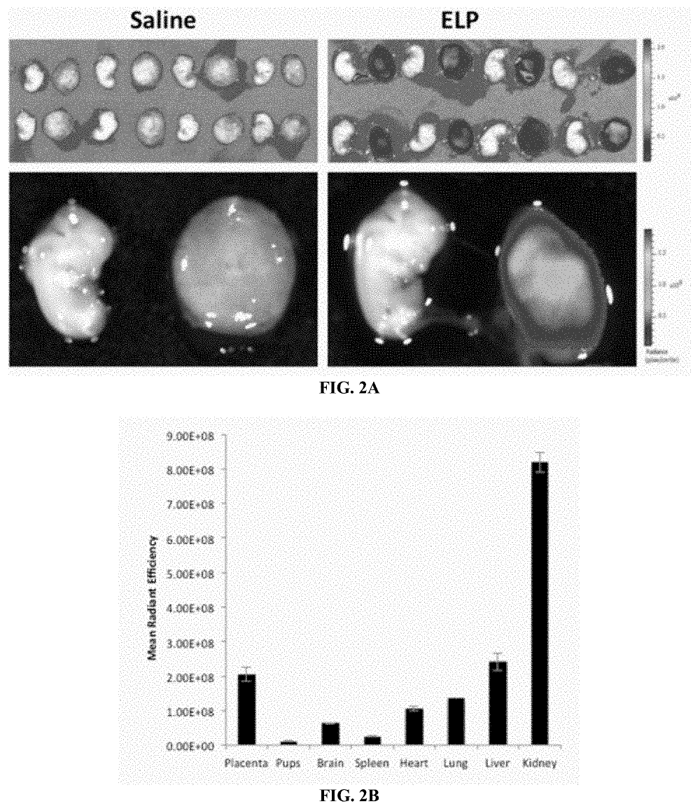

In order to test the hypothesis that ELP-fused therapeutics do not cross the placenta, an experiment was performed using the unmodified ELP carrier. Pregnant Sprague Dawley rats on day 14 of gestation were injected with fluorescently labeled ELP (100 mg/kg IV). Four hours after injection, which is about one half-life for this polypeptide, the rats were sacrificed and the placentas, pups, and major organs were removed for examination. Placentas and pups were dissected from the amniotic sacks and imaged ex vivo using an IVIS Spectrum animal imager to detect and quantitate the ELP levels. As shown in FIG. 2A, the placentas of animals injected with ELP-Alexa633 stained brightly, indicating that much protein had accumulated in them. In contrast, almost no ELP was detectable in the pups. The image intensities of the placentas, pups, and major organs were quantitated using Living Image software, and the results are shown in FIG. 2B. ELP accumulated strongly in the placenta, but ELP was barely detectable over autofluorescence in the pups. The placental accumulation was nearly equivalent to levels in the liver, an organ known to accumulate high levels of macromolecules due to its role in the reticuloendothelial system (Seymour L W, et al., 1987), and was second only to the kidney, which is likely actively involved in excretion and/or reuptake of the polypeptide. These high ELP levels in the placenta are unprecedented with this molecule. By way of comparison, the placenta levels are 2 to 10 fold higher than the tumors levels (Bidwell G L, et al., PloS One, 2013; Bidwell G L, 3rd, Cancer Lett., 2012). This is a reflection of the strong vascular perfusion of this organ and indicates that ELP is a great candidate carrier for placental drug delivery.

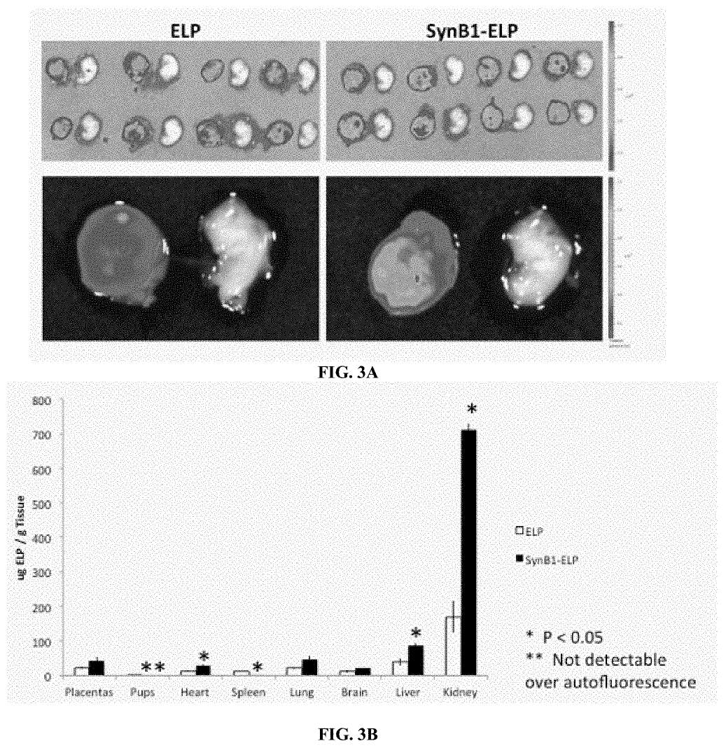

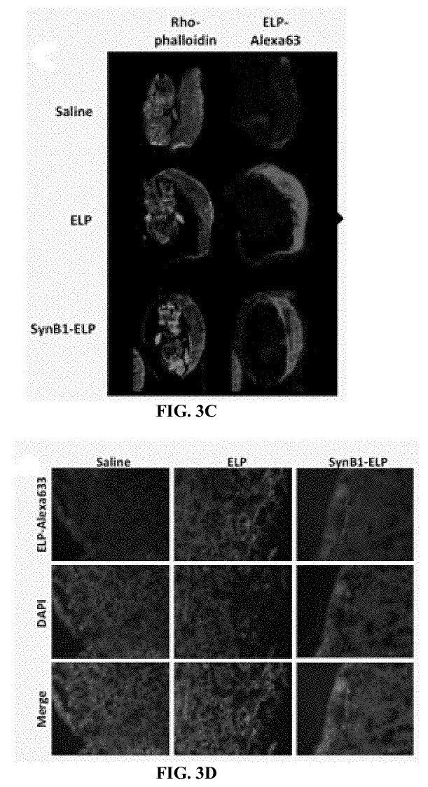

This example also examined whether the addition of a CPP to ELP would affect its penetration across the placenta. SynB1-ELP was labeled with Alexa633 and injected as described above. For comparison, animals were injected with saline control or ELP-Alexa633 at an equivalent dose. Four hours after injection, placental, fetal, and organ levels were determined by ex vivo fluorescence imaging. As shown in FIG. 3A, the addition of the SynB1 CPP to the ELP carrier increased its uptake in the placenta, but did not affect its delivery to the pups. FIG. 3B shows that the addition of SynB1 also increased the polypeptide deposition in the heart, liver, and kidneys relative to ELP, and SynB1 decreased deposition in the spleen. In addition to the ex vivo fluorescence imaging, the fetal-amino-placental complex was removed, rapidly frozen, and sectioned using a cryomicrotome. Tissue sections were stained with the actin-specific rhodamine-phalloidin to allow visualization of all tissue and imaged directly using a fluorescence slide scanner. As shown in FIG. 3C, both ELP-Alexa633 and SynB1-ELP-Alexa633 accumulated at high levels in the placenta relative to autofluorescence controls. In both cases, however, no polypeptide was detectable in the pups. The intraplacental distribution of SynB1-ELP-Alexa633 differed slightly from ELP-Alexa633. The unmodified ELP accumulated at high levels throughout the placenta, whereas SynB1-ELP accumulation was localized more strongly at the chorionic plate. Both polypeptides could be detected within the cells of the placenta when examined microscopically (FIG. 3D).

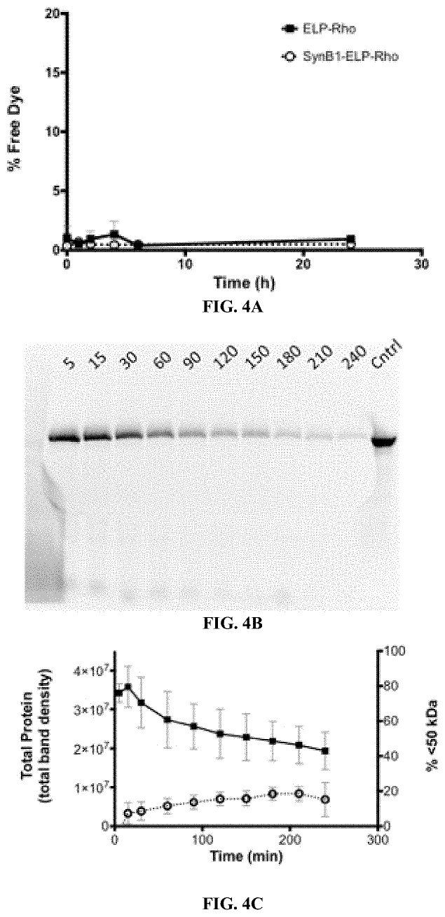

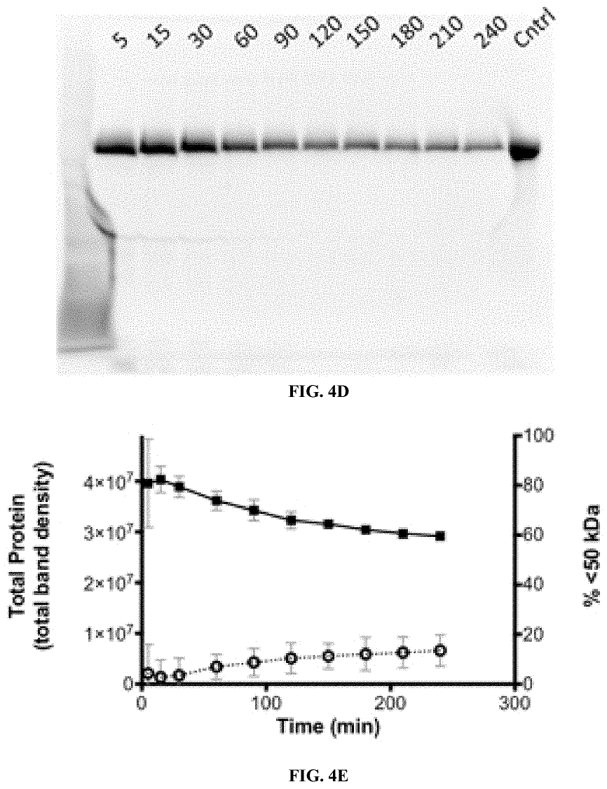

When using fluorescently labeled proteins, it is imperative that the label be stably bound in order to get accurate pharmacokinetic and biodistribution data. To determine the stability of the rhodamine label attached to the proteins via maleimide chemistry, the labeled protein is incubated in plasma from pregnant rats for various times at 37° C. After incubation, all proteins were precipitated using a 1:1 mixture with 10% trichloroacetic acid, and the fluorescence of the remaining supernatant was measured and compared to the pre-precipitation fluorescence. As shown in FIG. 4A, almost no label separated from the protein when incubated in rat plasma. Even after 24 h incubation, less than 2% of the dye was released from the protein. This demonstrates that the chemistry used to label these proteins is sufficient to produce a stable bond and confirms that the measurements are indeed of the labeled protein and not of released dye.

In addition to measuring dye release in vitro, the degradation of the protein in plasma samples in vivo is also examined. Plasma from the pharmacokinetic experiment above was analyzed by SDS-PAGE using direct fluorescence imaging to detect the labeled protein. As shown in FIGS. 4B and D, the total protein intensity decreased over time as the protein was cleared from circulation. However, very little protein degradation was apparent in either the ELP or the SynB1-ELP plasma samples. The amount of degradation was determined by measuring the total band intensity of the entire lane versus the total intensity of all bands <50 kDa. Plotting the total lane intensity (FIGS. 4C and E) revealed a clearance curve that closely overlayed the clearance seen with direct plasma fluorescence measurement. Analysis of the percentage of the band intensities at <50 kDa molecular weight revealed that very little degraded protein was present (right axis in FIGS. 4C and E). Even at the 4 hour time point, less than 20% of the total signal was present in these degraded bands. This analysis revealed that these proteins were quite stable in circulation.

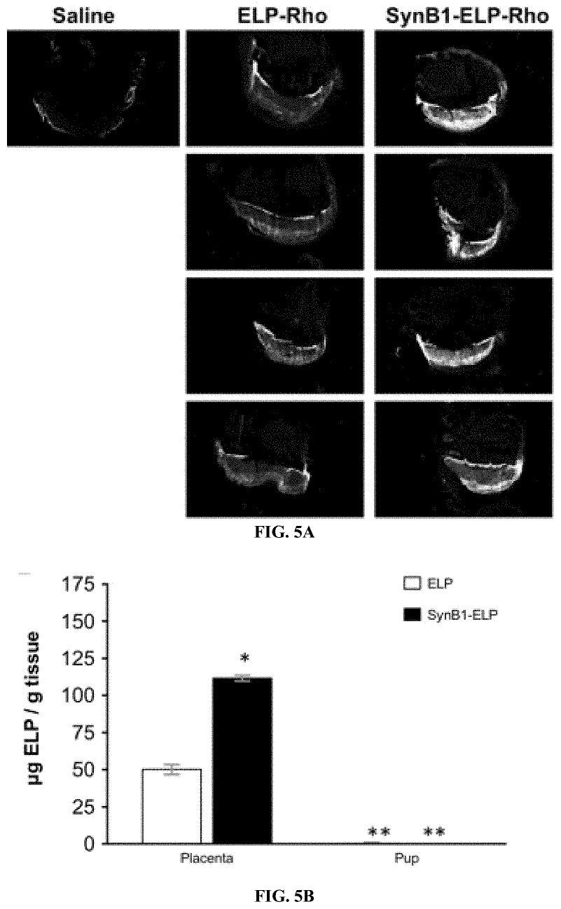

The ex vivo whole organ analysis shown in FIGS. 3A-D above gives a good snapshot of the polypeptide's biodistribution and an estimate of the actual tissue polypeptide levels. However, due to differences in organ size and therefore variability in the transmission of light through the tissue, combined with the difficulty of creating appropriate standards to correctly assess the absorbance and scattering of light, this technique has a limited ability to assess absolute tissue polypeptide levels. Therefore, quantitative fluorescence analysis of placental and pup polypeptide levels is also carried out using cryosections of intact feto-amino-placental units. By sectioning tissue and polypeptide standards to the same thickness, this technique allows for accurate quantitation of tissue polypeptide levels. As shown in FIG. 5A, this analysis confirmed that both polypeptides accumulated strongly in the placenta, but no polypeptide was detectable over autofluorescence in the pups. The images in FIG. 5A, all collected at the same scan settings, also indicate that SynB1-ELP accumulated at higher levels in the placenta than did ELP. The quantitative analysis revealed that ELP placental levels were approximately 50 μg/g of tissue (FIG. 5B). The placental level was increased over two-fold by the addition of the SynB1 CPP (p<0.0001). The quantitative fluorescence analysis also confirmed the fetal exclusion of both ELP and SynB1-ELP. Neither peptide was detectable in the pups using this method (FIG. 5B).

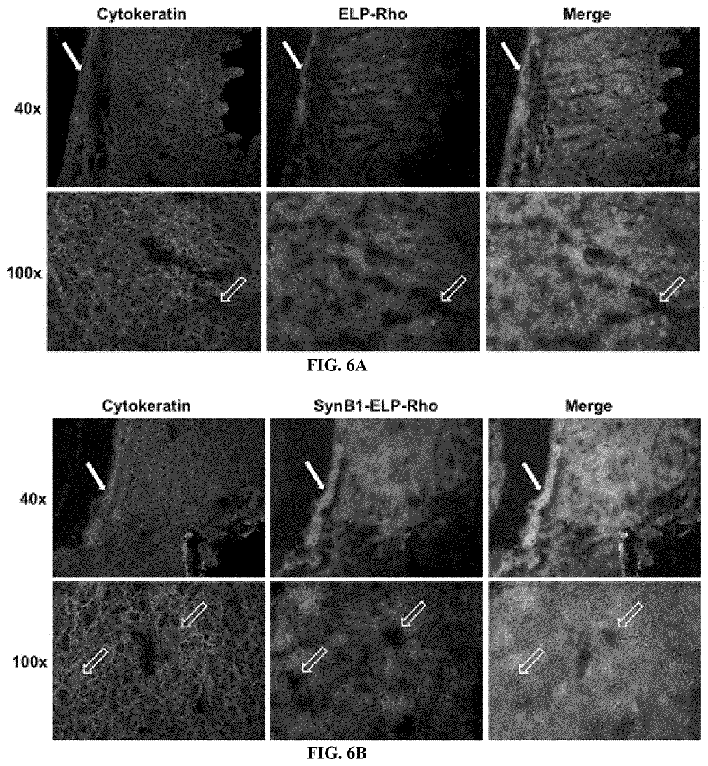

The placental tissue is also examined microscopically with a cytokeratin counterstain to detect trophoblast cells. Low magnification revealed that both ELP and SynB1-ELP accumulated highly at the chorionic plate (FIGS. 6A-B, solid arrows) and distributed diffusely within the labyrinth zone. Higher magnification revealed that both polypeptides accumulated in the cytoplasm of trophoblast cells. However, the interior of chorionic which contain fetal blood and are detected by voids in the cytokeratin staining, contained no ELP or SynB1-ELP (open arrows in FIGS. 6A-B). These results confirm at the cellular level the observations from the whole-organ and cryosection imaging that the ELP-based drug carrier is capable of entering cytotrophoblasts in the placenta but is excluded from transport into fetal circulation.

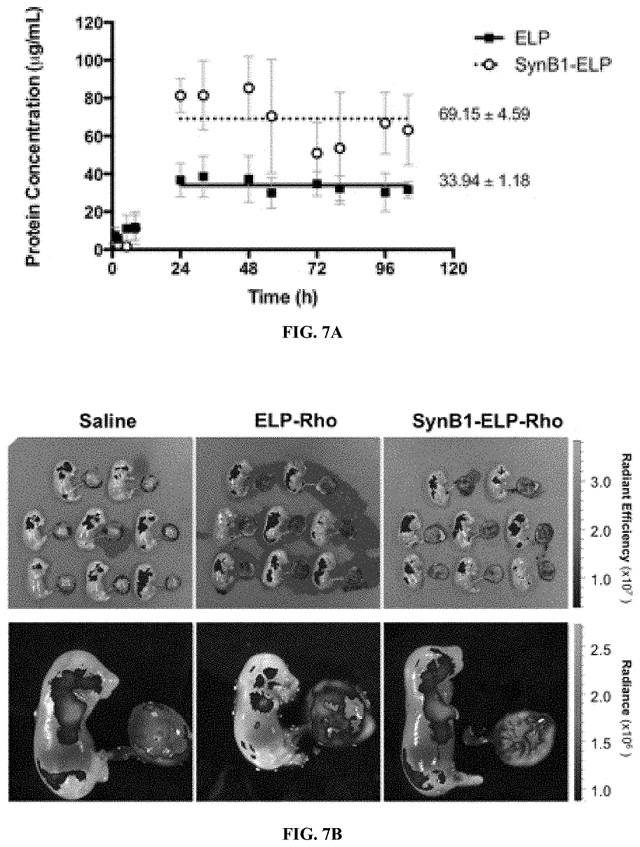

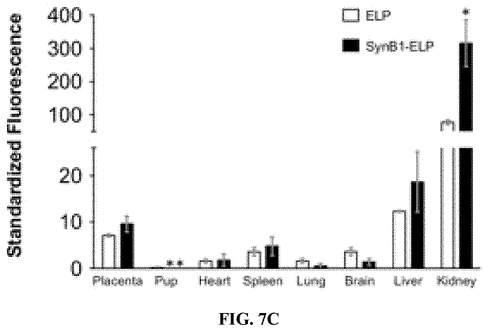

Ex vivo whole organ and quantitative histological fluorescence analysis revealed that ELP and SynB1-ELP accumulate highly in the placenta but are excluded from the fetus four hours after bolus administration on GD14. Whether the fetal exclusion held after five days of continuous infusion of the polypeptides is also examined. ELP or SynB1-ELP was administered continuously from GD14 to GD19 using an IP minipump. As shown in FIG. 7A, this technique lead to a steady state plasma level of the polypeptides beginning 24 h after pump implantation. At the dose used (30 mg/kg/day), the plasma levels were maintained at 33.94 μg/mL for ELP and 69.15 μg/mL for SynB1-ELP. These plasma levels are a reflection of many in vivo kinetic processes, including the rate of transport from the peritoneal fluid to the blood, the rate of extravasation from the blood to the tissues, and the plasma clearance rate. Since SynB1-ELP has a longer terminal plasma half-life than ELP, this likely explains why there is a higher steady-state level of SynB1-ELP in the plasma than ELP. These plasma concentration data also provide useful information for the formulation of dosages when ELP is fused with therapeutic agents.

Ex vivo whole organ fluorescence analysis of the placentas is performed, pups, and organs on GD19 following five days of continuous polypeptide infusion. Relative to the acute experiment, the placental levels of the polypeptides were lower, which resulted from the difference in dose (100 mg/kg in the bolus dosing versus 30 mg/kg/day in the chronic infusion). However, similar to the acute data, the polypeptides accumulated at high levels in the placenta but were undetectable over autofluorescence in the pups (FIG. 7B). The kidneys still accumulated the most polypeptide, followed by the liver and the placenta (FIG. 7C). Also, after chronic infusion, the effect of the CPP on the polypeptide biodistribution was much less pronounced. Only the kidneys contained significantly more SynB1-ELP than ELP (kidney levels of SynB1-ELP were increased four-fold relative ELP kidney levels, p=0.01). This indicates that the increases seen in the tissues immediately after infusion were the result of faster tissue deposition kinetics for SynB1-ELP relative to ELP, and after chronic administration, the tissue levels of the two polypeptides eventually became equivalent (with the exception of the kidneys).

In summary, this work has shown that the ELP and CPP-ELP carrier do not cross the placental barrier, even after five days of continuous infusion. These data demonstrate that a CPP can be used to direct intracellular delivery of the drug carrier within the placenta without affecting the penetration into the fetus.

### Example 4. Elp-Delivered Vegf

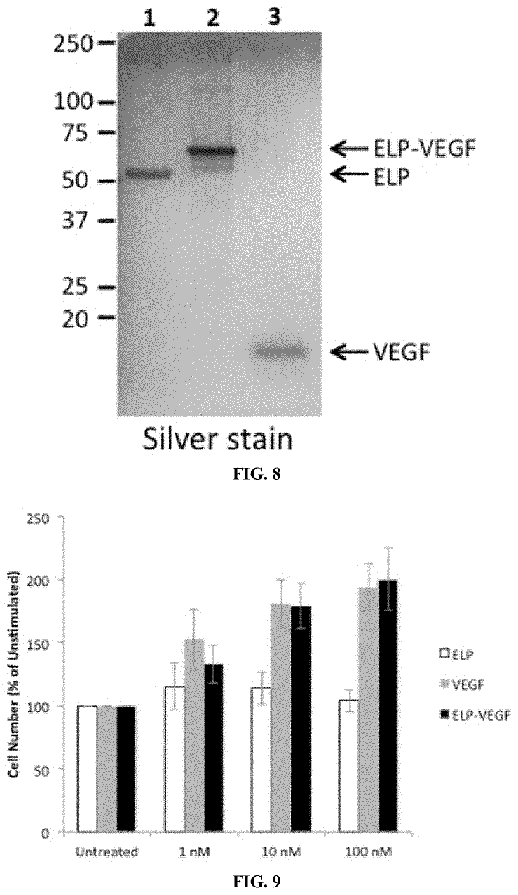

The coding sequence for VEGF was amplified from a human cDNA for VEGF-A. The sequence was modified by addition of C-terminal amino acids to generate a sequence identical to VEGF₁₂₁ in and to add restriction sites for cloning into the ELP expression vector. The coding sequence was cloned in frame with the ELP coding sequence to generate the ELP-VEGF chimeric construct. ELP-VEGF was expressed in E. coli BL21-Rosetta cells using the pET expression system with IPTG induction, and ELP-VEGF was purified by three to five rounds of inverse transition cycling (Bidwell G L, 3rd, et al., Mol Cancer Ther, 2005; Meyer D E, et al., 1999), taking advantage of the thermally responsive nature of ELP. The result was a 73 kDa protein that was very pure (FIG. 8).

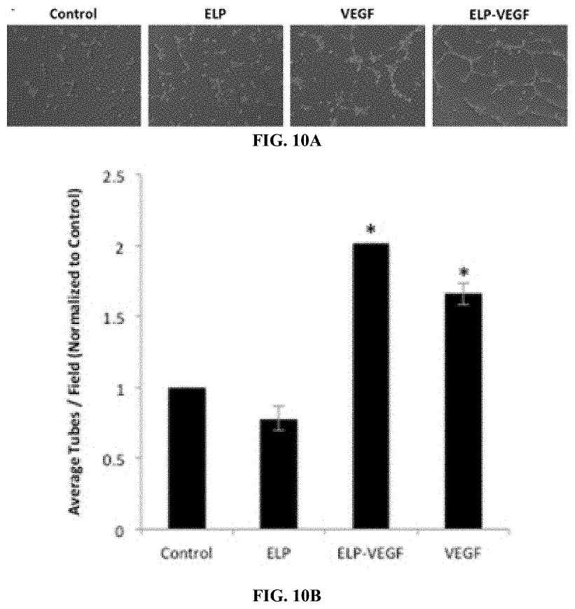

This example demonstrates that the ELP-VEGF was active and that ELP fusion did not alter the potency of VEGF. Proliferation of human umbilical vein endothelial cells (HUVECs) is stimulated when the cell are exposed to VEGF. As shown in FIG. 9, both free recombinant VEGF and ELP-VEGF stimulated HUVEC proliferation with equal potency. Furthermore, ELP-VEGF activated tube formation in HUVECs plated on growth factor reduced Matrigel, and the potency was again similar to or even slightly superior to that of free VEGF (FIGS. 10A-B). Finally, ELP-VEGF induced HUVEC migration in a Matrigel transwell cell migration assay with a similar potency as free VEGF (FIGS. 11A-B). These data indicate that a purified and highly potent ELP-VEGF chimeric protein is obtained, and the next phase of its preclinical testing is poised to carry out.

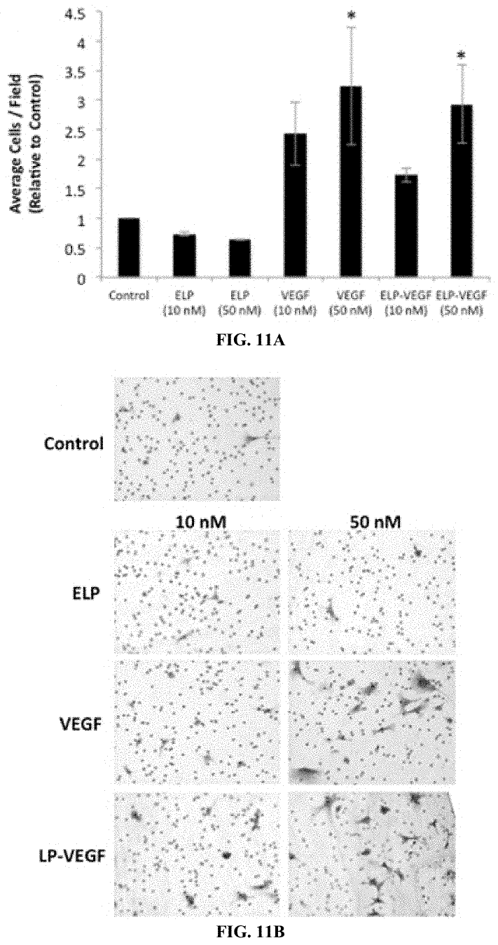

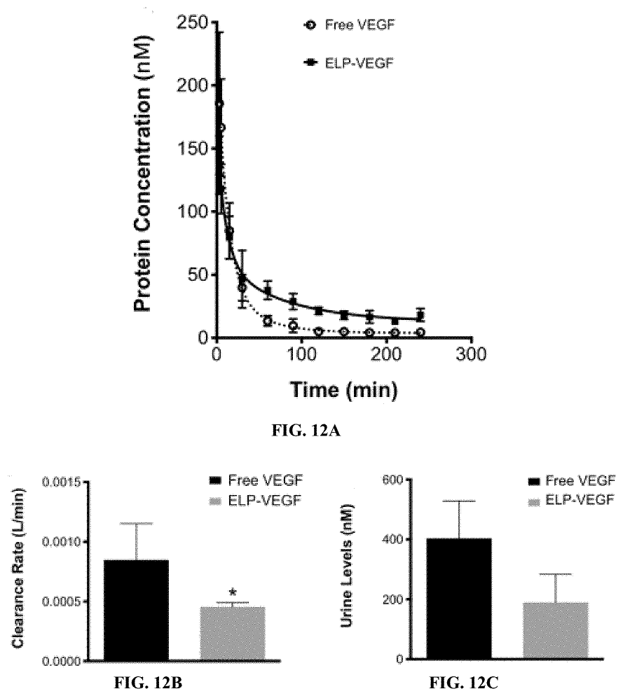

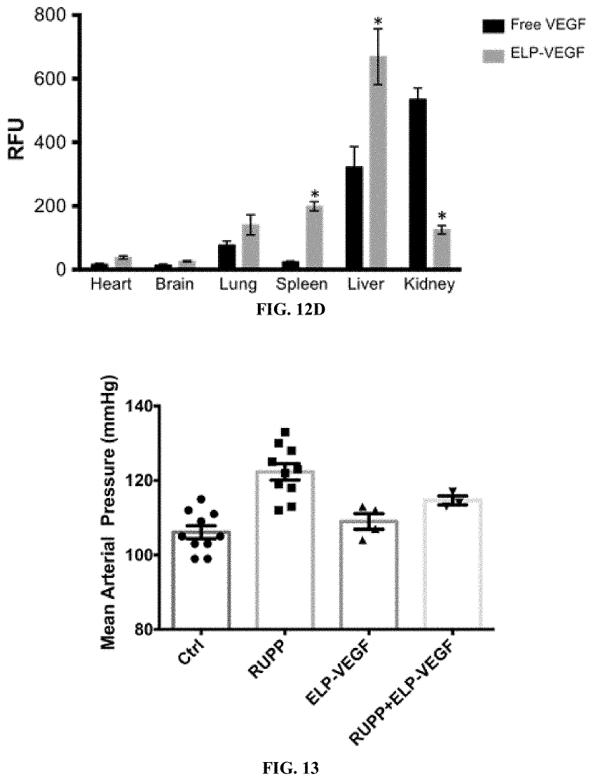

In addition to examining the ELP-VEGF activity in vitro, the pharmacokinetics (PK) and biodistribution of ELP-VEGF in comparison to free VEGF₁₂₁ is also determined. Both free VEGF₁₂₁ and ELP-VEGF were fluorescently labeled, and their PK and biodistribution were determined in mice after bolus intravenous administration. Free VEGF₁₂₁ had a very rapid plasma clearance (FIG. 12A), and fitting to a 2-compartment PK model revealed a terminal plasma half-life of approximately 30 minutes. This is consistent with other reports of approximately a 30 minute half-life for recombinant VEGF in humans. ELP-VEGF cleared more slowly than free VEGF (FIG. 12A). The plasma clearance rate of ELP-VEGF after IV infusion was about half the rate of free VEGF₁₂₁ (FIG. 12B), and as a result, there was less fluorescence detectable in the urine at the end of the experiment (FIG. 12C). Four hours after the infusion, the biodistribution was determined by ex vivo whole organ fluorescence imaging. VEGF₁₂₁ accumulated most highly in the kidneys and the liver and had very low levels in other organs. In contrast, ELP-VEGF accumulated more highly in the spleen and liver than did free VEGF₁₂₁, and the kidney deposition of ELP-VEGF was significantly lower than for free VEGF₁₂₁ (FIG. 12D).

Whether ELP-VEGF was effective for lowering blood pressure in a rat model of preeclampsia is texted next. Pregnant rats at gestational day 14 (GD14) were subjected to surgery to reduce the blood flow to the placentas. It has previously been shown that this model, achieved by partially restricting the ovarian arteries and the dorsal aorta, results in a preeclampsia-like syndrome in the rat. The effects mirror human preeclampsia in that the rats develop hypertension, proteinuria, reduced renal function, fetal growth restriction, and some fetal loss. The model also induces molecular markers that mirror the human syndrome, including elevated sFlt-1 levels, increased pro-inflammatory cytokines, and increased placental reactive oxygen species. The hypertension associated with this model can be seen in FIG. 13, where the mean arterial pressure increased from about 105 mmHg in normal pregnant rats on GD19 to over 120 mmHg in the preeclampsia model as measured by a pressure transducer inserted into a carotid arterial catheter. When ELP-VEGF was administered at a low dose of 1 mg/kg/day using an intraperitoneal minipump from GD14 to GD19, it effectively lowered the blood pressure of the preeclampsia-induced rats to near normal levels.

### Example 5. Elp-Delivered Nf-κB Inhibitory Peptide

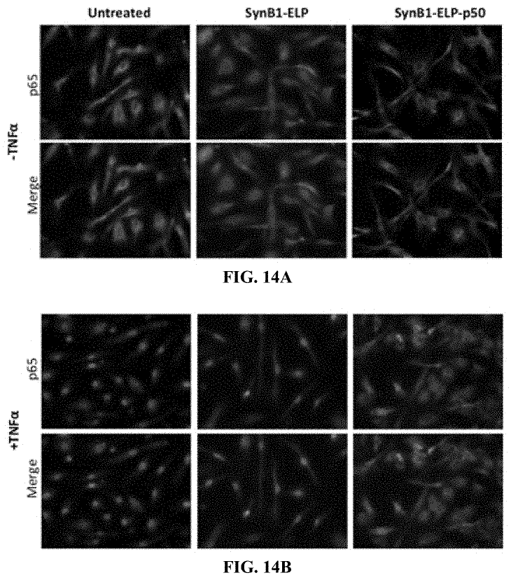

This investigation has developed an ELP-fused peptide inhibitor of activated NF-κB. NF-κB activation upon extracellular signaling is mediated by phosphorylation and release of the natural inhibitor I-κB from the NF-κB p50/p65 heterodimer. I-κB release exposes a nuclear localization sequence (NLS) on the p50 subunit of NF-κB, and once exposed, this NLS mediates nuclear import of NF-κB. Once inside the nucleus, NF-κB binds to response elements on its target genes and regulates gene expression. A synthetic cell permeable peptide containing the p50 NLS is capable of blocking the nuclear import of NF-κB upon stimulation in a variety of cell lines (Lin Y Z, et al., 1995). A copy of the p50 NLS is fused to the SynB1-ELP carrier and validated its activity using an in vitro NF-κB activation assay. Stimulation of cultured HUVECs with TNF-α leads to rapid activation of the NF-κB pathway, and this can be detected by monitoring nuclear localization of NF-κB (FIG. 14A, left panel). As shown in FIGS. 14A-B, pretreatment of the cells with SynB-ELP-p50, but not the SynB1-ELP control polypeptide, completely blocks this nuclear translocation of NF-κB (FIG. 14A, middle and right panels, quantified in 30-60 cells/sample in FIG. 14B).

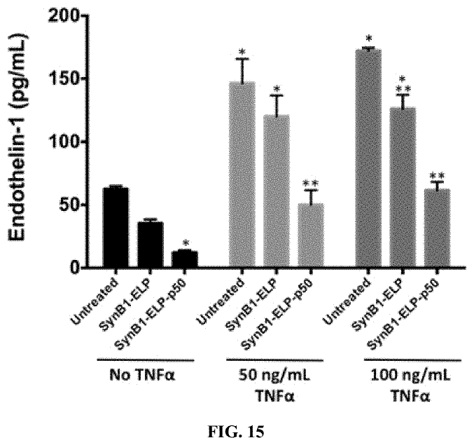

TNFα stimulation also leads to the secretion of the vasoactive peptide endothelin-1 by HUVECs. This endothelin release contributes to the hypertension associated with the pro-inflammatory environment in preeclampsia. As shown in FIG. 15, endothelin levels in the culture media increase about three-fold when the cells are stimulated with TNFα. However, when the cells are pre-treated with the SynB1-ELP-p50 peptide, the endothelin release is completely blocked. In addition, SynB1-ELP-p50 decreases the endothelin release from unstimulated HUVECs.

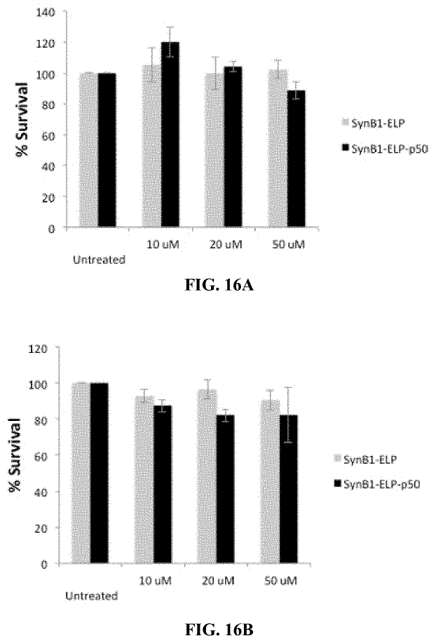

To test whether the NF-κB inhibitory polypeptide had any effect on proliferation of normal tissue cell types, were determined its effects on proliferation of endothelial and chorionic cells. As shown in FIGS. 16A-B, HUVEC endothelial cells and BeWo chorionic cells were exposed to the indicated concentrations of SynB1-ELP or SynB1-ELP-p50 for 72 h, and cell number was determined by MTS assay. Neither SynB1-ELP or SynB1-ELP-p50 had any detectable effect on proliferation of HUVEC or BeWo cells at concentrations up to 50 μM. These data indicate that an active NF-κB inhibitory polypeptide with potent anti-inflammatory and anti-hypertensive properties and low cytotoxicity has been synthesized and purified.

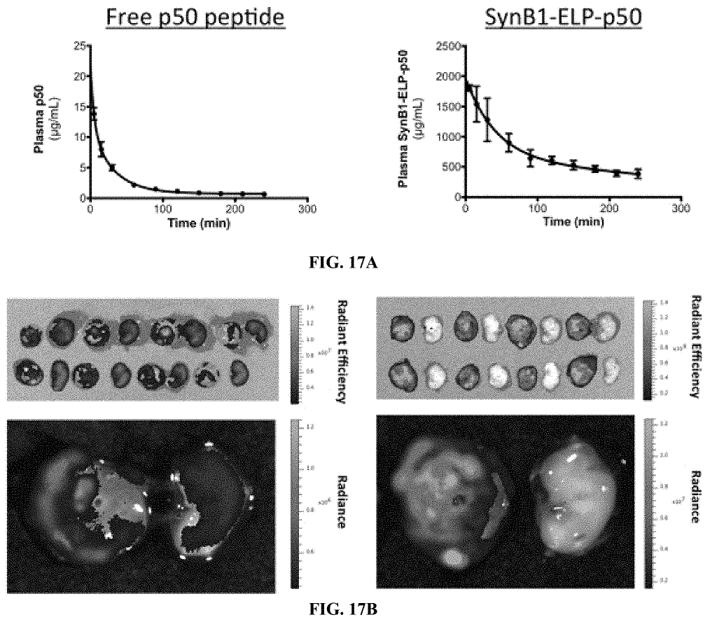

Using pregnant Sprague Dawley rats, the pharmacokinetics and biodistribution of the SynB1-ELP-delivered p50 peptide with the free p50 peptide is determined. Rats were given a single bolus dose of 100 mg/kg of rhodamine-labeled SynB1-ELP-p50 or free p50, blood was sampled intermittently for four hours, and organs, placentas, and pups were removed for ex vivo fluorescence analysis. As shown in FIGS. 17A-B, SynB1-ELP delivery had massive effects in the pharmacokinetics and the biodistribution of the p50 peptide. Initial plasma levels of the p50 peptide were about 100-fold lower than SynB1-ELP-p50 levels, indicated very rapid clearance of the majority of the injected peptide. Also, when the plasma clearance data were fit to a two-compartment pharmacokinetic model, the p50 peptide cleared with a terminal half-life of 21 minutes, whereas SynB1-ELP-p50 had a terminal half-life of greater than two hours (FIG. 17A). Placenta and pups levels were determined four hours after injection by ex vivo fluorescence analysis. As shown in FIG. 17B, total placental levels of the free p50 peptide were thirty fold lower than placental SynB1-ELP-p50 levels. Importantly, in addition to vastly lower therapeutic levels in the placenta, the unconjugated p50 peptide freely entered the fetal circulation and was visible in the pups. These data indicate that ELP fusion greatly enhances the plasma half-life and tissue levels of a therapeutic peptide, and it effectively prevents the peptide from entering the fetal circulation.

### Example 6. Elp-Delivered Nadph Oxidase Inhibitory Peptide

The cell penetrating NADPH oxidase inhibitory polypeptide was generated by modifying the coding sequence for ELP with the addition of the coding sequence for the SynB1 CPP at its N-terminus and with the coding sequence for the NOX inhibitory peptide at its C-terminus. A DNA cassette encoding the SynB1 and NOX peptides separated by an SfiI restriction site and containing sticky ends compatible with NdeI and BamHI restriction sites was synthesized (Integrated DNA Technologies). The cassette was cloned into pET25b between the NdeI and BamHI restriction sites. The coding sequence for ELP was restricted from its pUC19 host vector using PflMI and BglI, the DNA was gel purified, and it was ligated into the SfiI site of the modified pET25b vector. The result was an in-frame fusion of SynB1, ELP, and the NOX peptide (SynB1-ELP-NOX). The final construct was confirmed by DNA sequencing and transformed into the BLR (DE3) expression strain (Novagen). A construct containing the SynB1 peptide fused to the N-terminus of ELP, but lacking the NOX peptide (SynB1-ELP) was generated in a similar manner. Polypeptides were purified by three to five rounds of inverse transition cycling.

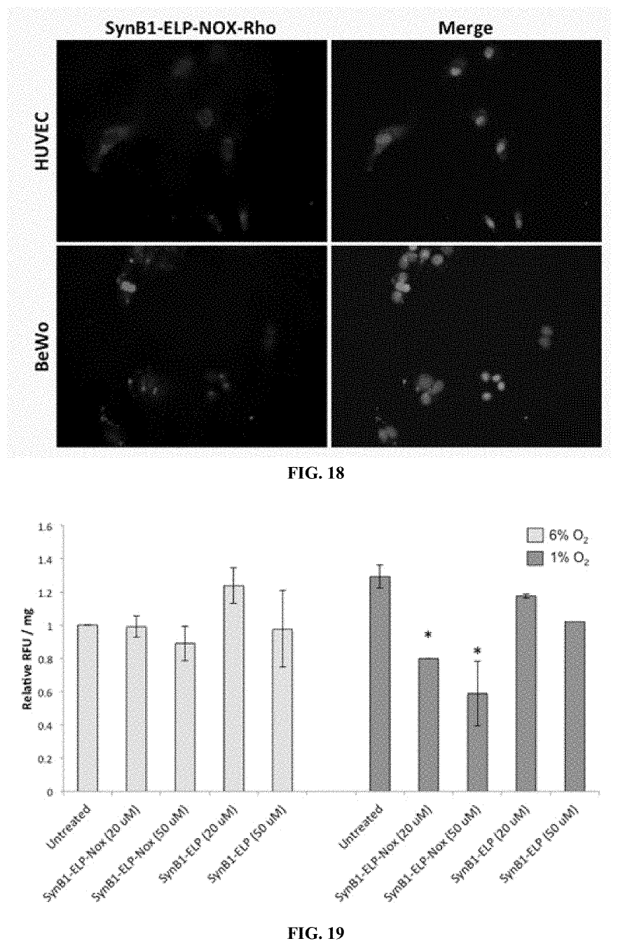

It is confirmed that the SynB1-ELP-NOX polypeptide was internalized by cells. Both endothelial cells (HUVECs) and chorionic cells (BeWo choriocarcinoma cells) were exposed to fluorescently labeled SynB1-ELP-NOX for 1 h. The cells were then washed and given fresh media for 24 h. Internalization was confirmed by fluorescence microscopy as shown in FIG. 18. The SynB1-ELP-NOX polypeptide was detectable in a punctate cytoplasmic distribution in both cell lines.

Next, the ability of the SynB1-ELP-NOX polypeptide to block ROS production in placental chorionic villous explants is demonstrated. Chorionic villous explants were cut on GD19 and cultured ex vivo on Matrigel coated wells with complete cell culture medium. After equilibration, culture medium was replaced with medium containing SynB1-ELP-NOX or the SynB1-ELP control polypeptides at 20 or 50 μM, and explants were incubated at 6% O₂ (representing a healthy placenta) or 1% O₂ (representing a preeclamptic placenta). After 48 h exposure to hypoxia, detection of ROS was performed using the dihydroethidium (DHE) assay. As shown in FIG. 19, explants cultured at 1% O₂ produced more ROS than explants cultured at 6% O₂. Incubation with SynB1-ELP-NOX inhibited this ROS production and even resulted in ROS levels in hypoxic explants that were lower than normoxic controls. The SynB1-ELP polypeptide that lacks the NOX inhibitory domain had no effect on ROS production. These results demonstrate that SynB1-ELP-NOX can block NADPH oxidase induces ROS production, and they show the promise of this agent for therapy of ROS-driven diseases.

### Example 7. Using Cell Penetrating Peptides and Organ Targeting Peptides to Direct ELP's Biodistribution

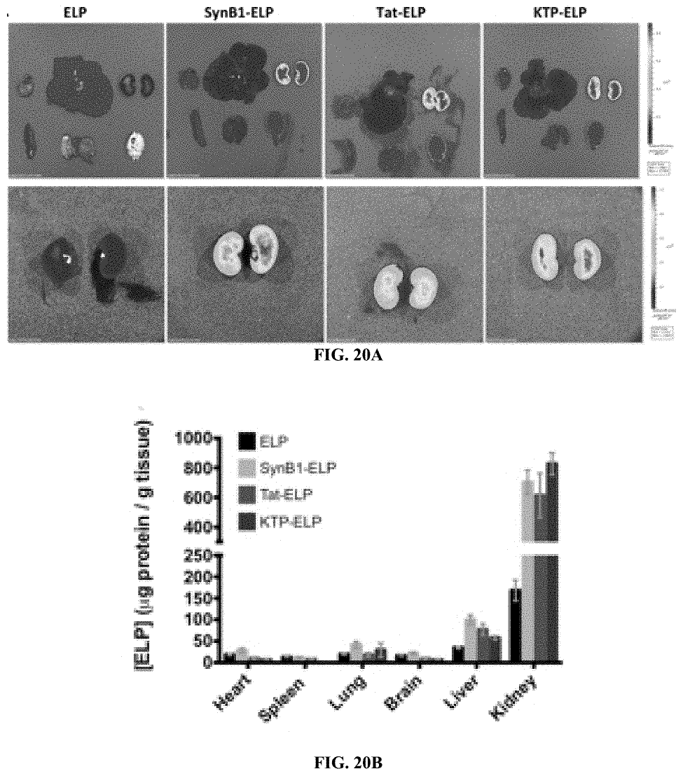

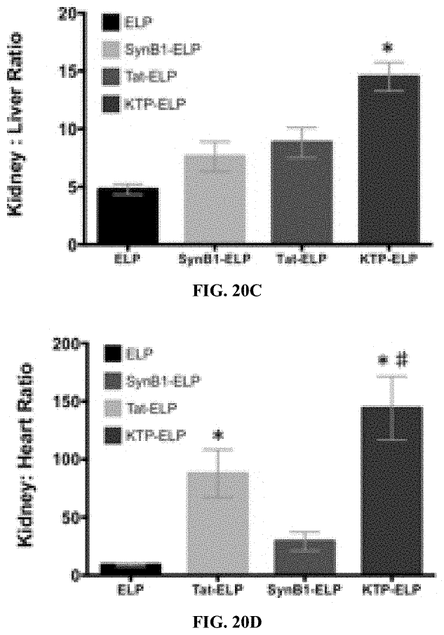

For various diseases, it is often beneficial to deliver therapeutics to specific organs of interest. Organ targeting can increase the efficacy of the delivered therapeutic, and it can reduce off-target side effects. For treatment of preeclampsia, which mediated by factors produced in the placenta which act in systemic vascular beds and in the kidney, it would be beneficial to deliver pro-angiogenic, anti-inflammatory, or anti-oxidant therapeutics to both the placenta and the kidneys. ELP naturally accumulates at high levels in the kidneys, and FIGS. 3A-B and 17A-B above show that ELP or a CPP-fused ELP accumulates at high levels in the placenta. Here, it is sought to optimize the kidney targeting by testing multiple CPPs and by testing a peptide designed to target the vascular endothelium of the kidney (kidney targeting peptide (KTP)). Sprague Dawley rats were administered ELP, the CPP-fused ELPs Tat-ELP and SynB1-ELP, or the kidney targeting peptide-fused KTP-ELP by bolus IV injection. Plasma was sampled intermittently for four hours, then organs were removed and analyzed by ex vivo fluorescence imaging. The CPPs or KTP did not have dramatic effects on the plasma clearance rate of ELP (not shown), but the peptides did dramatically alter the biodistribution. All three peptides increased ELP deposition in the kidney by over five-fold (FIGS. 20A-B). Also, the CPPs Tat and SynB1 increased ELP deposition in the liver. When kidney specificity was assessed by measuring kidney: liver (FIG. 20C) and kidney: heart (FIG. 20D) ratios, KTP was found to be the most specific peptide for targeting the kidney (inducing a three-fold enhancement of ELP levels relative to the liver and over 15-fold enhancement relative to the heart). These data demonstrate that cell penetrating peptides and organ targeting peptides can be employed to direct the biodistribution of the ELP drug carrier.

### Examples 8-11

Examples 8-11 are directed to demonstrating the effects of molecular weight on the pharmacokinetics, biodistribution, and placenta deposition of ELP and defining the molecular weights best suited for placental drug delivery depending on the intra-placenta target. More specifically, Examples 8-11 show that in addition to an increased half-life and tissue accumulation with an increase in their molecular weights, specific sized ELP constructs were surprisingly and unexpectedly found to target only specific regions of the placenta. Alternatively, other sized ELP constructs simultaneously target multiple regions of the placenta. Beyond the specific application to placental drug delivery, these Examples also provide a detailed characterization of how ELP chain length affects the protein's pharmacokinetics and biodistribution during pregnancy, which is critical information when developing ELPs as drug carriers for other diseases and conditions of pregnancy.

ELP is a genetically engineered polypeptide consisting of repeated units of a five amino-acid motif, and it has a unique physical property called thermal responsiveness. Above a characteristic transition temperature, the polypeptide forms aggregates, while below the transition temperature, the aggregates re-dissolve. There are many advantages of using ELP for drug delivery. First, ELPs are genetically encoded rather than chemically synthesized. This means the user has absolute control over the ELP sequence and molecular weight (MW), and it allows the addition of targeting peptides and therapeutic peptides. Second, ELP and ELP-fusion proteins can be expressed in E. coli or other recombinant expression systems, allowing large quantities of the molecules to be purified easily because the polypeptide is thermally responsive. Purification of ELP-fusion proteins is achieved by heating a lysate containing the recombinantly expressed ELP above the polypeptides' transition temperature. This induces ELP aggregation, and it is collected by centrifugation. Repeated centrifugation above and below the transition temperature leads to large quantities of very pure protein. The third advantage of using ELP for drug delivery is that it is a large, non-immunogenic macromolecule. Therefore, ELP fusion can stabilize small protein or peptide or small molecule therapeutic agent cargo in systemic circulation, and targeting agents can be used to direct the ELP-fused therapeutics' biodistribution.

Starting with ELP, it was coupled to the therapeutic agent that may be a peptide or protein or protein fragment or small molecule drug known to have therapeutic activity in preeclampsia or other pregnancy related disease or condition. In addition to altering the physical properties of the ELP carrier itself, other attributes of the ELP coupled therapeutic agent are designed. To optimize the drug delivery to the placenta, in vivo targeting may be accomplished by the inclusion of targeting sequences or peptides on the ELP carrier coupled to the targeting agent. The targeting agent may be a peptide, protein, or small molecule with a specific molecular target in the placenta. Further, it also may also contain a cell penetrating peptide, other peptide, or protein capable of penetrating the cellular membrane.

Other modifications of the drug delivery system included a drug binding domain to allow attachment of known or new small molecule therapeutic agents to improve their delivery to treat preeclampsia and other pregnancy related disorders or to treat other diseases that happen to occur during pregnancy such as cancer. The drug binding domain may be attached to the ELP carrier via a drug release domain to allow for selective release of the drug under particular environmental conditions or at specific sites within the body. In other delivery vehicles, the ELP coupled therapeutic system includes multiple copies of the therapeutic agent and/or drug binding domain to increase the amount of drug delivered. This may also include the use of 2 or more different therapeutic agents or different drugs attached to the drug binding domain/s to achieve combination therapy. Other cases may include both a therapeutic agent/s and a drug binding domain/s to achieve simultaneous delivery of peptide/protein-based therapeutic agents with small molecule drugs.

### Example 8: Pharmacokinetic Study of an ELP Proteins with Varying Molecular Weights in Pregnant Rats

The prior patent application described the use of the ELP drug delivery system for maternally sequestered drug delivery and for prevention of fetal drug exposure. In these embodiments, a library of ELPs with varying MW were evaluated (Table 4). The predicted molecular weight in the table are based on the primary amino acid sequence (the number following “ELP” in the protein name represents the number of repeats of SEQ ID NO: 1), as calculated by the ExPASy ProtParam tool. Transition temperature was measured by turbidity analysis. Hydrodynamic radius was measured by dynamic light scattering (adapted from Kuna, et al., Scientific Reports, 2018 May 21; 8(1):7923). The MW of the ELP protein has strong effects on the polymer's hydrodynamic radius, pharmacokinetics, and biodistribution.

TABLE 4
Size and Transition Temperature of the
ELP Constructs Used in this Example.

| | Predicted | Transition | |
|---|---|---|---|
| | protein MW | temperature | Radium |
| Protein | (kDa) | (° C.) | (nm) |
| ELP-63 | 25.2475 | 89.745 | 4.170 ± 0.056 |
| ELP-127 | 49.5469 | 65.775 | 5.800 ± 0.200 |
| ELP-223 | 85.996 | 60.250 | 6.830 ± 0.169 |

A pharmacokinetic study was conducted in Sprague Dawley timed pregnant rats on gestational day 14 (GD14) to determine the effects of MW on plasma clearance of ELPs. This time point was chosen as it represents the first day of the third week of the rodent gestation, which is a commonly used time point to deliver therapeutics in rat models of preeclampsia drug development studies.

Animal studies were approved by the Animal Care and Use Committee of the University of Mississippi Medical Center and conducted according to the guidelines of the Guide for the Care and Use of Laboratory Animals. For pharmacokinetic and biodistribution experiments, three ELPs ranging in MW from 25 kDa (63 repeats of SEQ ID NO: 1) to 86 kDa (223 repeats of SEQ ID NO: 1) were used. Sprague Dawley timed pregnant rats on GD14 (Charles River) were anesthetized with isoflurane (1-3%, to effect) and injected with rhodamine-labeled polypeptides (1.5 μmol/kg) by intravenous injection into the femoral vein. Blood was sampled by tail prick at various time points for 4 hours, collected in Greiner Bio-One MiniCollect capillary blood collection tubes (Greiner Bio-One), and plasma was collected after centrifugation. Plasma samples were analyzed for concentration of the polypeptides using quantitative fluorescence analysis.

Plasma ELP levels were assessed with a two-way repeat measures ANOVA for factors of polypeptide treatment and time with a post hoc Tukey's multiple comparison. Organ biodistribution was assessed with a two-way ANOVA for factors of polypeptide treatment and organ type with post hoc Tukey's multiple comparison. The relationship between MW and organ levels was determined by Pearson correlation coefficient. Urine free dye and rhodamine levels were assessed by a one-way ANOVA for differences in polypeptide treatment with post hoc Tukey's multiple comparison. In all analyses, a p value of <0.05 was considered statistically significant. All statistical analysis and curve fitting was done using GraphPad Prism (Version 7.04).

The preparation of ELP proteins for the study was performed as follows. The synthesis of ELP expression constructs was performed by recursive directional ligation. pET25b+vectors encoding ELP proteins were transformed into E. coli BLR (DE3). All proteins were purified by inverse transition cycling. Fluorescent labeling of ELP proteins was accomplished by labeling each ELP protein on its N-terminal cysteine residue using a maleimide conjugate of rhodamine.

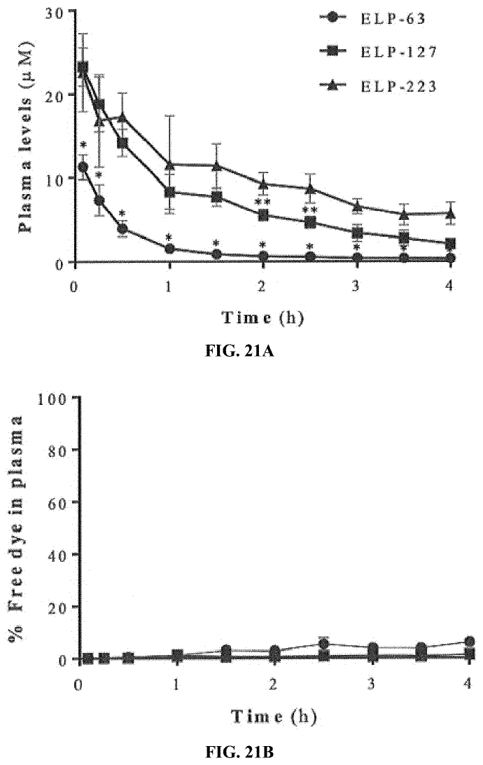

After preparation, three ELPs with MWs of 25, 50, and 86 kDa were administered by bolus IV injection. Blood was sampled at various time points up to 4 h after bolus i.v. injection. Based on direct fluorescence measurement of plasma, an increase in MW of these proteins resulted in slower plasma clearance (FIG. 21A). All of the proteins used had the highest plasma concentration immediately after bolus i.v. injection at 5 min time point. ELP-63 plasma levels were lower than ELP-127 and ELP-223 at all time points tested, and ELP-127 plasma levels were lower than ELP-223 plasma levels at the 2 h and 2.5 h time points. At the four hour time point, the smallest protein ELP-63 (25 kDa) levels were lowest at 0.35±0.06 μM, while ELP-127 (50 kDa) was 2.09±0.45 μM, and the largest protein ELP-223 (86 kDa) was 5.66±1.35 μM.

The trends in plasma clearance are consistent with our observations for these proteins in non-pregnant mice, in which we measured 48 hours of plasma clearance data. In FIG. 21B, the effects of fluorophore loss from polypeptides were assessed by measuring the plasma fluorescence before and after precipitation of the proteins with TCA. Values are mean SD, n=4, *ELP-63 levels significantly different from ELP-127 and ELP-223, **ELP-127 levels significantly different from ELP-223 as assessed by a two-way repeated measures ANOVA with post hoc Tukey's multiple comparison, p<0.05. Further analysis by TCA precipitation revealed that the fluorescence present in the plasma was not free fluorophore (FIG. 21B). None of the proteins tested had more than 6% free dye in the plasma.

### Example 9: Biodistribution and Localization of Different MW ELP Proteins in Organs of a Rat Pregnancy Model

For tissue biodistribution studies, the same animals from the pharmacokinetic study were euthanized while still under anesthesia. The placenta, pups and major organs were collected and measured by whole organ fluorescence imaging and biodistribution analysis (n=4 rats per agent). Major organs and placentae and their associated pups (n>5 per animal) were imaged ex vivo using an IVIS Spectrum. Total organ fluorescence was quantified and fit to standard curves of the appropriate ELP to correct for any differences in labeling levels among polypeptides.

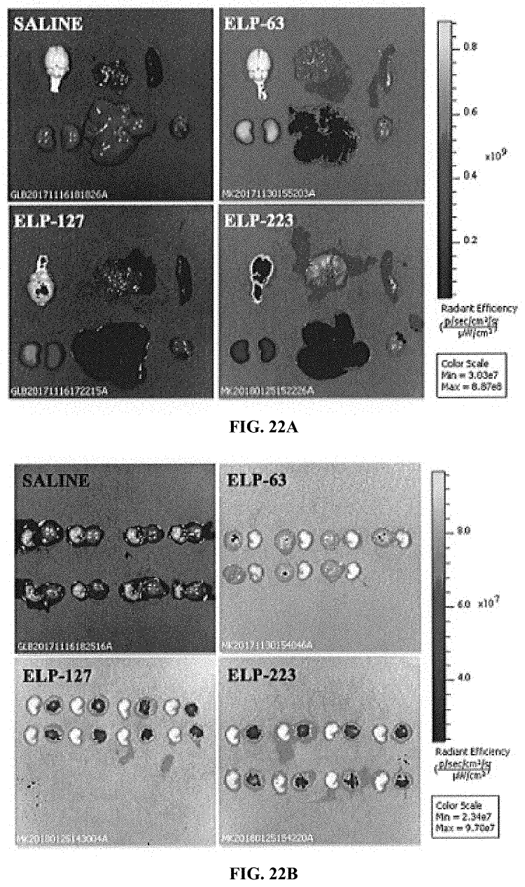

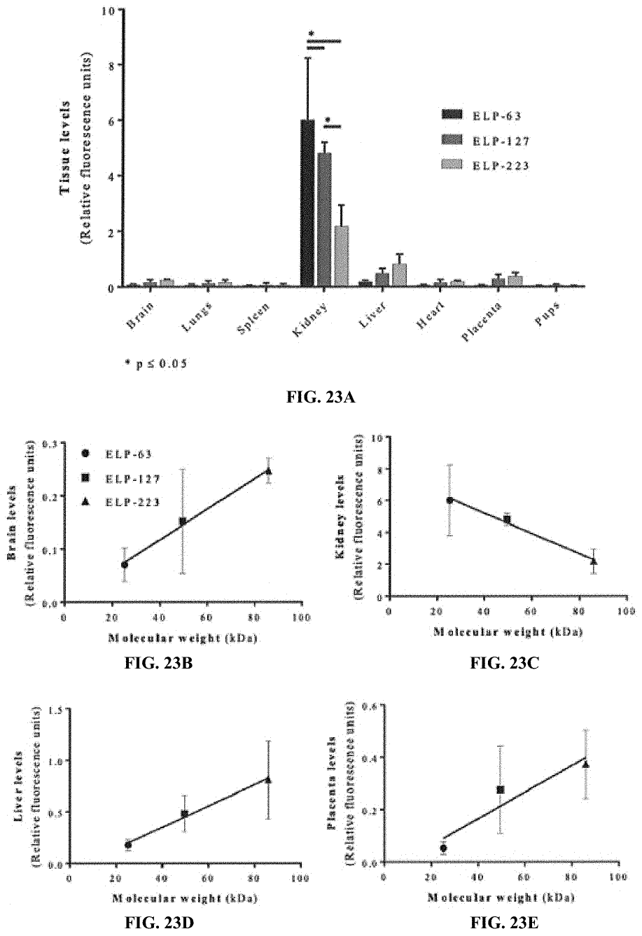

Representative images of major organs, placentae, and pups for each of the treatment groups are shown in FIGS. 22A-B. In FIG. 22A, a representative image of major organs from one animal from each group and in FIG. 22B, a representative image of placentae and corresponding pups from one animal from each group. The high renal accumulation of all ELPs tested is apparent in FIG. 23A. FIGS. 23A and E show that there is also strong ELP signal in the placenta, especially for ELP-127 and ELP-223, while there is no detectable ELP in the pups.

Tissue biodistribution of ELP constructs in a rat pregnancy model was examined as follows. The change in fluorescence levels in the tissue accumulation of labeled ELPs having varying MW was determined using data was quantified relative to standard curves of the injection protein using the images in FIGS. 22A-B.

The ELP proteins used in the study accumulated strongly in the kidneys regardless of their MW, followed by the liver and placenta (FIGS. 23A-E). The relationship between MW and organ levels was determined using a Pearson correlation coefficient, and the results of the analysis reported in Table 5, as R² and p value for three xy pairs.

TABLE 5
Results of Correlation Analysis for MW and Organ Levels.

| | r | R² | P value |
|---|---|---|---|
| Brain | 0.9973 | 0.9946 | 0.0468 |
| Lungs | 0.9282 | 0.8615 | 0.2427 |
| Spleen | 0.495 | 0.245 | 0.6703 |
| Kidney | −0.9948 | 0.9897 | 0.0648 |
| Liver | 0.9953 | 0.9907 | 0.0615 |
| Heart | 0.9341 | 0.8726 | 0.2324 |
| Placenta | 0.9436 | 0.8904 | 0.2148 |
| Pups | 0.255 | 0.06503 | 0.8358 |

ELP levels in individual major organs was examined as a function of MW were fit by linear regression using GraphPad Prism. In FIGS. 23B-E, florescence values are mean±SD, n=4, of at least five pups per rat. Statistically significant difference (*) between indicated groups as assessed by a two-way ANOVA with post hoc Tukey's multiple comparison, p<0.05. Brain, lungs, liver, heart and placenta ELP levels had a positive correlation with ELP MW, with Pearson correlation coefficients r>0.9. In contrast, the kidneys levels had a negative correlation with MW with an r=−0.9. For all organs, the R² was >0.8, and while a trend for correlation was apparent, only the brain correlation had a p value <0.05 (FIG. 23B). These results showed that tissue levels of ELP were dependent on the ELP MW, with brain, liver and placenta showing trends for increased ELP deposition with increasing MW and the kidney showing a trend for decreased accumulation with increasing MW.

### Example 10: Urine Analysis

Urine samples collected prior to euthanasia four hours after protein injection were analyzed by quantitative fluorescence analysis. The fluorescence intensity of 2 μl of urine was measured in a plate reader before and after trichloroacetic acid (TCA) precipitation. Post-precipitation levels were corrected for dilution and compared to pre-precipitation fluorescence to calculate percentage of free dye. Urine samples were analyzed for creatinine levels using QuantiChrom Creatinine Assay Kit (DICT-500, BioAssay Systems) per Manufacturer's instructions. Urine samples were also analyzed by SDS-PAGE. 10 μl of each urine sample was prepared with Bolt LDS sample buffer 4× (Novex, Thermo Fisher Scientific), analyzed on a Bolt 4-12% Bis-tris Plus gel, and visualized by direct fluorescence imaging using an IVIS Spectrum (PerkinElmer) using 535-nm excitation and 580-nm emission filters and small binning, followed by Coomassie Brilliant Blue staining.

Urine was collected from the animals at the time of organ harvest, four hours after bolus i.v. injection of fluorescently labeled proteins before and after precipitation of the proteins with TCA. Urine fluorescence was fit to a standard curve, corrected for rhodamine and creatinine concentration, and assessed by one-way ANOVA with post hoc Tukey's multiple comparison (F(2, 9)=5.706, p=0.0251). Rhodamine levels in the urine corrected by creatinine were significantly different between ELP-127 and ELP-223, as assessed by one-way ANOVA with post hoc Tukey's multiple comparison (FIG. 24A). Fluorescence was corrected for slight differences in injection volume, fit to a standard curve, and normalized by creatinine level.

Analysis of fluorescence before and after TCA precipitation revealed that the urine contained 78, 72, and 69% free fluorophore for ELP-63, ELP-127, and ELP-223 (FIG. 24B), respectively. These levels were not significantly different (one-way ANOVA with post hoc Tukey's multiple comparison (F(2, 9)=2.994, p=0.1007). There was no correlation between ELP MW and either urine fluorescence levels or percent free dye (Pearson's correlation coefficient for urine % free dye and rhodamine concentration corrected by creatinine had a negative value of −0.9594 and −0.8121, respectively. R² values were 0.9205 and 0.6595, and p=0.1819 and 0.3967, respectively).

SDS-PAGE analysis of urine samples with direct fluorescence imaging is shown in FIG. 24C and Coomassie staining FIG. 24D. Values are mean±SD, n=4. *Statistically significant difference between indicated groups as assessed by one-way ANOVA with post hoc Tukey's multiple comparison, p<0.05.

Gel electrophoresis with direct fluorescence detection revealed that no intact ELP was present in the urine (FIG. 24C). A major band was present at the bottom of the gel, representing free rhodamine, and a less intense band was apparent just above the free rhodamine band likely indicating dye bound to an amino acid or very short peptide fragment. These results indicate that no ELP passes intact to the urine, and they are consistent with our previous findings in mice that ELPs are strongly reabsorbed in the kidneys by the tubular protein reuptake system. A Coomassie stain revealed a similar banding pattern for all rats, with no bands correlating to full length ELP and similar loading levels among lanes (though the levels of an ˜60 kDa protein, likely albumin. did vary from rat to rat).

### Example 11: Placental Localization of ELP Proteins

To investigate the localization of different MW ELP proteins in the placenta after administration, sections of intact feto-amnio-placental units were imaged by confocal microscopy. One amniotic sac, containing a pup and its placenta, was removed from each animal and kept intact. These feto-amnio-placental units were then embedded in freezing medium (Tissue-Plus O.C.T Compound) and flash frozen in isopentane on dry ice. Placentae and pups were cut into 20 μm sections with a cryostat. Kidneys were also embedded, flash frozen and cut into 14 μm sections. Slides were equilibrated to room temperature, and unprocessed tissue sections were imaged by confocal microscopy image stitching using a 561-nm laser and a 10× magnification objective. The same imaging settings were maintained for all samples. Brightness was adjusted equally among all groups to allow for better visualization while still maintaining quantitative differences in fluorescence levels.

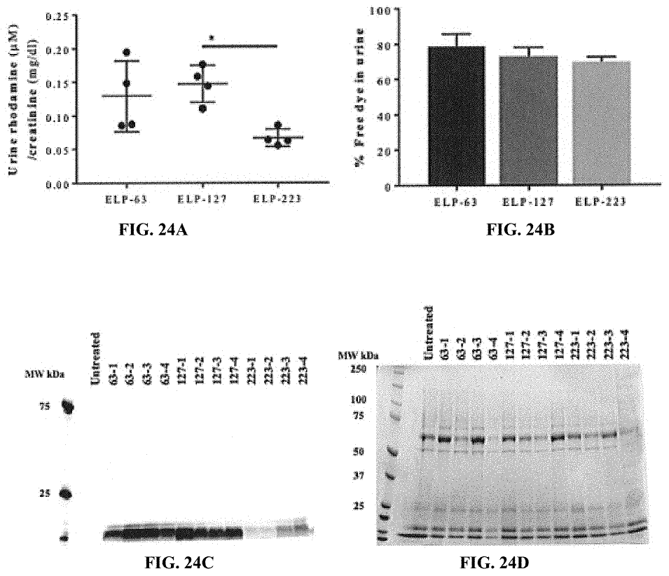

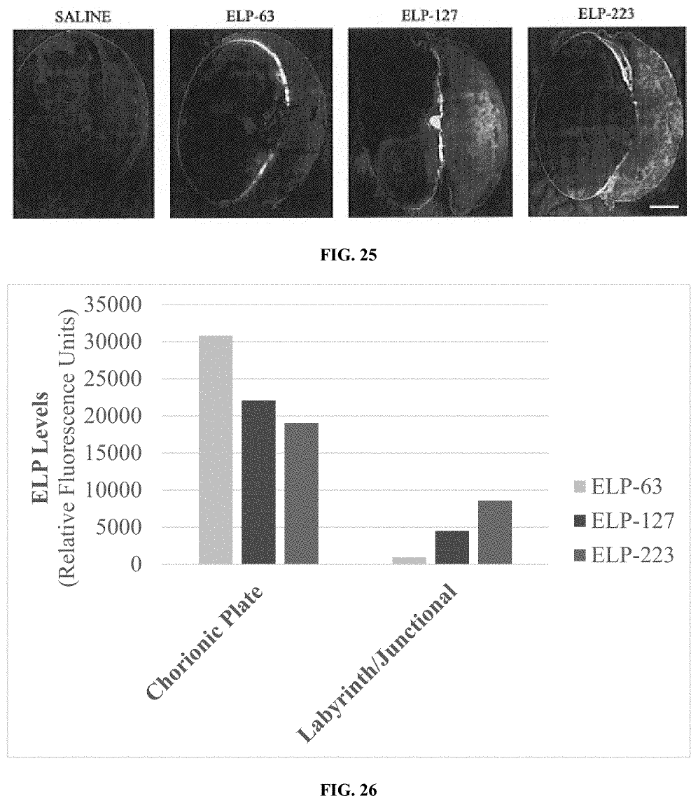

In FIG. 25, placental localization of ELPs in placental sections obtained by confocal microscopy image stitching, scale bar=2000 μm. Imaging parameters were identical among all samples, and image intensities represent actual protein levels. There was an increase in placental levels with an increase in MW as shown in FIG. 25. In all cases, no ELP signal was detected in the fetus.

It is demonstrated that, due to an increase in ELP plasma half-life combined with the large blood pool in the placenta, accumulation of ELP carriers in maternal organs, especially in the placenta, is increased with an increase in ELP MW. Surprisingly, as shown in FIG. 26, localization within the placenta is MW dependent, and intra-placental localization is achieved by tuning the ELP MW size where smaller MW ELP proteins, such as ELP-63, reside predominantly in the chorionic plate while larger MW ELP proteins, such as ELP-127 and ELP-223, are present in the labyrinth and junctional zone with increasing MW.

For quantitative measurements, intra-placental ELP levels were determined from whole-slice imaging of intact feto-amnio-placental units. Not only were the overall placental levels dependent on the size of the ELP protein, the distribution within the placenta also varied by ELP size. As shown in FIG. 26, based on relative fluorescent units, the smallest ELP tested, containing 63 repeats of SEQ ID NO: 1 (ELP-63), localized almost exclusively to the chorionic plate. Of total relative fluorescent units, 97% of ELP-63 resided in the chorionic plate, with less than 3% localized to the labyrinth and junctional zones. As the ELP size increased, distribution in the chorionic plate decreased and distribution in both the labyrinth and junctional zones increased. For the largest ELP tested, ELP-223, 69% of the protein was localized in the chorionic plate, and 31% was distributed in the labyrinth and junctional zones. ELP-127 had approximately 17% present in labyrinth and junctional zones and 83$ in the chorionic plate.

In conclusion, all of the ELPs had significant accumulation at the chorionic plate, however the ELP-63 was found to be predominantly localized in the chorionic plate and not present throughout the other placenta regions. For the delivery of at least 90% of therapeutics to the chorionic plate of the placenta, an ELP including 70 repeat units of SEQ ID NO: 1 or less, about 30 kDaltons in size, is required. In contrast, larger MW ELPs, ELP-127 and ELP-223, were distributed throughout the labyrinth and junctional zone rather than being only in the chorionic plate region. The largest MW ELP, ELP-223, having the highest placental level was broadly distributed throughout the labyrinth and junctional zone, indicating that larger MW ELP have increasing levels in the labyrinth and junctional zone. To provide a therapeutic of least 15% distribution to the labyrinth and junctional zones of the placenta, the preferred ELP requires 95 repeat units of SEQ ID NO: 1 or a size of about 37 kDaltons or greater. The differences will play a major role in delivering adequate amounts of the therapeutic to the critical areas of the placenta.

Throughout this document, various references are mentioned. All such references are incorporated herein by reference, including the references set forth in the following list.

### REFERENCES

Clark D E, Smith S K, He Y, et al. A vascular endothelial growth factor antagonist is produced by the human placenta and released into the maternal circulation. Biology of reproduction. December 1998; 59(6):1540-1548.
2. Banai S, Jaklitsch M T, Shou M, et al. Angiogenic-induced enhancement of collateral blood flow to ischemic myocardium by vascular endothelial growth factor in dogs. Circulation. May 1994; 89(5):2183-2189.
3. Pearlman J D, Hibberd M G, Chuang M L, et al. Magnetic resonance mapping demonstrates benefits of VEGF-induced myocardial angiogenesis. Nat Med. October 1995; 1(10):1085-1089.
4. Chade A R, Kelsen S. Reversal of renal dysfunction by targeted administration of VEGF into the stenotic kidney: a novel potential therapeutic approach. American journal of physiology. Renal physiology. May 15, 2012; 302(10):F1342-1350.
5. Chade A R. VEGF: Potential therapy for renal regeneration. F1000 medicine reports. 2012; 4:1.
6. Cvetanovic M, Patel J M, Marti H H, Kini A R, Opal P. Vascular endothelial growth factor ameliorates the ataxic phenotype in a mouse model of spinocerebellar ataxia type 1. Nat Med. 2011; 17(11):1445-1447.
Eppler S M, Combs D L, Henry T D, et al. A target-mediated model to describe the pharmacokinetics and hemodynamic effects of recombinant human vascular endothelial growth factor in humans. Clinical pharmacology and therapeutics. July 2002; 72(1):20-32.
8. Miquerol L, Langille B L, Nagy A. Embryonic development is disrupted by modest increases in vascular endothelial growth factor gene expression. Development. September 2000; 127(18):3941-3946.
9. Drake C J, Little C D. Exogenous vascular endothelial growth factor induces malformed and hyperfused vessels during embryonic neovascularization. Proc Natl Acad Sci USA. Aug. 15, 1995; 92(17):7657-7661.
10. He Y, Smith S K, Day K A, Clark D E, Licence D R, Charnock-Jones D S. Alternative splicing of vascular endothelial growth factor (VEGF)-R1 (FLT-1) pre-mRNA is important for the regulation of VEGF activity. Mol Endocrinol. April 1999; 13(4):537-545.
11. Makarov S S. NF-kappa B in rheumatoid arthritis: a pivotal regulator of inflammation,
hyperplasia, and tissue destruction. Arthritis research. 2001; 3(4):200-206.
12. Lin Y Z, Yao S Y, Veach R A, Torgerson T R, Hawiger J. Inhibition of nuclear translocation of transcription factor NF-kappa B by a synthetic peptide containing a cell membrane-permeable motif and nuclear localization sequence. J Biol Chem. Jun. 16, 1995; 270(24):14255-14258.
13. Paravicini T M, Touyz R M. NADPH oxidases, reactive oxygen species, and hypertension: clinical implications and therapeutic possibilities. Diabetes care. February 2008; 31 Suppl 2:S170-180.
14. Park Y M, Febbraio M, Silverstein R L. CD36 modulates migration of mouse and human macrophages in response to oxidized LDL and may contribute to macrophage trapping in the arterial intima. J Clin Invest. January 2009; 119(1):136-145.
15. Radermacher K A, Wingler K, Langhauser F, et al. Neuroprotection After Stroke by Targeting NOX4 As a Source of Oxidative Stress. Antioxidants & redox signaling. Apr. 20, 2013; 18(12):1418-1427.
16. Matsubara S, Sato I. Enzyme histochemically detectable NAD(P)H oxidase in human placental trophoblasts: normal, preeclamptic, and fetal growth restriction-complicated pregnancy. Histochemistry and cell biology. July 2001; 116(1):1-7.
17. Khan S R. Reactive oxygen species as the molecular modulators of calcium oxalate kidney stone formation: evidence from clinical and experimental investigations. J Urol. March 2013; 189(3):803-811.
18. Block M L. NADPH oxidase as a therapeutic target in Alzheimer's disease. BMC neuroscience. 2008; 9 Suppl 2:S8.
19. Wang X, Pinto-Duarte A, Sejnowski T J, Behrens M M. How Nox2-Containing NADPH Oxidase Affects Cortical Circuits in the NMDA Receptor Antagonist Model of Schizophrenia. Antioxidants & redox signaling. Apr. 20, 2013; 18(12):1444-1462.
20. Whitehead N P, Yeung E W, Froehner S C, Allen D G. Skeletal muscle NADPH oxidase is increased and triggers stretch-induced damage in the mdx mouse. PloS one. 2010; 5(12):e15354.
21. Monaghan-Benson E, Hartmann J, Vendrov A E, et al. The role of vascular endothelial growth factor-induced activation of NADPH oxidase in choroidal endothelial cells and choroidal neovascularization. Am J Pathol. October 2010; 177(4):2091-2102.
22. Araneda O F, Tuesta M. Lung oxidative damage by hypoxia. Oxidative medicine and cellular longevity. 2012; 2012:856918.
23. Cifuentes-Pagano E, Csanyi G, Pagano P J. NADPH oxidase inhibitors: a decade of discovery from Nox2ds to HTS. Cell Mol Life Sci. July 2012; 69(14):2315-2325.
24. Csanyi G, Cifuentes-Pagano E, Al Ghouleh I, et al. Nox2 B-loop peptide, Nox2ds, specifically inhibits the NADPH oxidase Nox2. Free Radic Biol Med. Sep. 15, 2011; 51(6):1116-1125.
25. Moktan S, Perkins E, Kratz F, Raucher D. Thermal targeting of an acid-sensitive doxorubicin conjugate of elastin-like polypeptide enhances the therapeutic efficacy compared with the parent compound in vivo. Mol Cancer Ther. July 2012; 11(7):1547-1556.
26. Moktan S, Ryppa C, Kratz F, Raucher D. A thermally responsive biopolymer conjugated to an acid-sensitive derivative of paclitaxel stabilizes microtubules, arrests cell cycle, and induces apoptosis. Invest New Drugs. Oct. 12, 2010.
27. Bidwell G L, Perkins E, Hughes J, Khan M, James J, Raucher D. Thermally Targeted Delivery of a c-Myc Inhibitory Polypeptide Inhibits Tumor Progression and Extends Survival in a Rat Glioma Model. PloS one. 2013; In Press.
28. Bidwell G L, 3rd, Perkins E, Raucher D. A thermally targeted c-Myc inhibitory polypeptide inhibits breast tumor growth. Cancer Lett. Jun. 28, 2012; 319(2):136-143.
29. Urry D W, Parker T M, Reid M C, Gowda D C. Bioact Compat Polym. 1991; 6(3):263-282.
30. Bidwell G L. Peptides for Cancer Therapy—A Drug Development Opportunity and a Drug Delivery Challenge. Therapeutic Delivery. 2012; in press.
31. Bidwell G L, 3rd, Raucher D. Application of thermally responsive polypeptides directed against c-Myc transcriptional function for cancer therapy. Mol Cancer Ther. July 2005; 4(7):1076-1085.
32. Bidwell G L, 3rd, Raucher D. Cell penetrating elastin-like polypeptides for therapeutic peptide delivery. Adv Drug Deliv Rev. Dec. 30, 2010; 62(15):1486-1496.
33. Bidwell G L, 3rd, Whittom A A, Thomas E, Lyons D, Hebert M D, Raucher D. A thermally targeted peptide inhibitor of symmetrical dimethylation inhibits cancer-cell proliferation. Peptides. May 2010; 31(5):834-841.
34. Massodi I, Bidwell G L, 3rd, Raucher D. Evaluation of cell penetrating peptides fused to elastin-like polypeptide for drug delivery. J Control Release. Nov. 28, 2005; 108(2-3):396-408.
35. Massodi I, Thomas E, Raucher D. Application of thermally responsive elastin-like polypeptide fused to a lactoferrin-derived peptide for treatment of pancreatic cancer. Molecules. 2009; 14(6):1999-2015.
36. Meyer D E, Chilkoti A. Purification of Recombinant Proteins by Fusion with Thermally Responsive Polypeptides. Nat. Biotechnol. 1999; 17:1112-1115.
37. Moktan S, Raucher D. Anticancer activity of proapoptotic peptides is highly improved by thermal targeting using elastin-like polypeptides. International journal of peptide research and therapeutics. September 2012; 18(3):227-237.
38. Bidwell G L, 3rd, Davis A N, Raucher D. Targeting a c-Myc inhibitory polypeptide to specific intracellular compartments using cell penetrating peptides. J Control Release. Apr. 2, 2009; 135(1):2-10.
39. Luan C H, Parker T M, Gowda D C, Urry D W. Hydrophobicity of amino acid residues: differential scanning calorimetry and synthesis of the aromatic analogues of the polypentapeptide of elastin. Biopolymers. September 1992; 32(9):1251-1261.
40. Luan C H, Parker T M, Prasad K U, Urry D W. Differential scanning calorimetry studies of NaCl effect on the inverse temperature transition of some elastin-based polytetra-, polypenta-, and polynonapeptides. Biopolymers. April 1991; 31(5):465-475.
41. Urry D W, Long M M, Cox B A, Ohnishi T, Mitchell L W, Jacobs M. The synthetic polypentapeptide of elastin coacervates and forms filamentous aggregates. Biochim Biophys Acta. Dec. 18, 1974; 371(2):597-602.
42. Meyer D E, Kong G A, Dewhirst M W, Zalutsky M R, Chilkoti A. Targeting a Genetically Engineered Elastin-like Polypeptide to Solid Tumors by Local Hyperthermia. Cancer Res. Feb. 15, 2001 2001; 61(4):1548-1554.
43. Meyer D E, Shin B C, Kong G A, Dewhirst M W, Chilkoti A. Drug targeting using thermally responsive polymers and local hyperthermia. J Control Release. Jul. 6, 2001; 74(1-3):213-224.
44. Dreher M R, Raucher D, Balu N, Michael Colvin O, Ludeman S M, Chilkoti A. Evaluation of an elastin-like polypeptide-doxorubicin conjugate for cancer therapy. J Control Release. Aug. 28, 2003; 91(1-2):31-43.
45. Massodi I, Moktan S, Rawat A, Bidwell G L, 3rd, Raucher D. Inhibition of ovarian cancer cell proliferation by a cell cycle inhibitory peptide fused to a thermally responsive polypeptide carrier. Int J Cancer. Jan. 15, 2010; 126(2):533-544.
46. Furgeson D Y, Dreher M R, Chilkoti A. Structural optimization of a “smart” doxorubicin-polypeptide conjugate for thermally targeted delivery to solid tumors. J Control Release. Jan. 10, 2006; 110(2):362-369.
47. Bidwell G L, 3rd, Davis A N, Fokt I, Priebe W, Raucher D. A thermally targeted elastin-like polypeptide-doxorubicin conjugate overcomes drug resistance. Invest New Drugs. August 2007; 25(4):313-326.
48. Bidwell G L, 3rd, Fokt I, Priebe W, Raucher D. Development of elastin-like polypeptide for thermally targeted delivery of doxorubicin. Biochem Pharmacol. Mar. 1, 2007; 73 (5):620-631.
49. Massodi I, Bidwell G L, 3rd, Davis A, et al. Inhibition of ovarian cancer cell metastasis by a fusion polypeptide Tat-ELP. Clin Exp Metastasis. 2009; 26(3):251-260.
50. Liu W, Mackay J A, Dreher M R, et al. Injectable intratumoral depot of thermally responsive polypeptide-radionuclide conjugates delays tumor progression in a mouse model. J Control Release. Jan. 31, 2010; in press.
51. Liu W, McDaniel J, Li X, et al. Brachytherapy using injectable seeds that are self-assembled from genetically encoded polypeptides in situ. Cancer Res. Nov. 15, 2012; 72(22):5956-5965.
52. MacKay J A, Chen M, McDaniel J R, Liu W, Simnick A J, Chilkoti A. Self-assembling chimeric polypeptide-doxorubicin conjugate nanoparticles that abolish tumours after a single injection. Nat Mater. December 2009; 8(12):993-999.
53. Simnick A J, Amiram M, Liu W, et al. In vivo tumor targeting by a NGR-decorated micelle of a recombinant diblock copolypeptide. J Control Release. Oct. 30, 2011; 155(2):144-151.
54. Na K, Lee S A, Jung S H, Hyun J, Shin B C. Elastin-like polypeptide modified liposomes for enhancing cellular uptake into tumor cells. Colloids and surfaces. B, Biointerfaces. Mar. 1, 2012; 91:130-136.
55. Callahan D J, Liu W, Li X, et al. Triple stimulus-responsive polypeptide nanoparticles that enhance intratumoral spatial distribution. Nano letters. Apr. 11, 2012; 12(4):2165-2170.
56. McDaniel J R, Macewan S R, Dewhirst M, Chilkoti A. Doxorubicin-conjugated chimeric polypeptide nanoparticles that respond to mild hyperthermia. J Control Release. May 10, 2012; 159(3):362-367.
57. Walker L, Perkins E, Kratz F, Raucher D. Cell penetrating peptides fused to a thermally targeted biopolymer drug carrier improve the delivery and antitumor efficacy of an acid-sensitive doxorubicin derivative. Int J Pharm. Oct. 15, 2012; 436(1-2):825-832.
58. Betre H, Setton L A, Meyer D E, Chilkoti A. Characterization of a genetically engineered elastin-like polypeptide for cartilaginous tissue repair. Biomacromolecules. September-October 2002; 3(5):910-916.
59. McHale M K, Setton L A, Chilkoti A. Synthesis and in vitro evaluation of enzymatically cross-linked elastin-like polypeptide gels for cartilaginous tissue repair. Tissue engineering. November-December 2005; 11(11-12):1768-1779.
60. Shamji M F, Chen J, Friedman A H, Richardson W J, Chilkoti A, Setton L A. Synthesis and characterization of a thermally-responsive tumor necrosis factor antagonist. J Control Release. Aug. 7, 2008; 129(3):179-186.
61. Shamji M F, Jing L, Chen J, et al. Treatment of neuroinflammation by soluble tumor necrosis factor receptor Type II fused to a thermally responsive carrier. Journal of neurosurgery. Spine. August 2008; 9(2):221-228.
62. Moss I L, Gordon L, Woodhouse K A, Whyne C M, Yee A J. A novel thiol-modified hyaluronan and elastin-like polypetide composite material for tissue engineering of the nucleus pulposus of the intervertebral disc. Spine. June 2011; 36(13):1022-1029.
63. Dreher M R, Elas M, Ichikawa K, et al. Nitroxide conjugate of a thermally responsive elastin-like polypeptide for noninvasive thermometry. Med Phys. October 2004; 31(10):2755-2762.
64. Chen T H, Bae Y, Furgeson D Y. Intelligent biosynthetic nanobiomaterials (IBNs) for hyperthermic gene delivery. Pharm Res. March 2008; 25(3):683-691.
65. Conrad U, Plagmann I, Malchow S, et al. ELPylated anti-human TNF therapeutic single-domain antibodies for prevention of lethal septic shock. Plant biotechnology journal. January 2011; 9(1):22-31.
66. Na K, Jung J, Lee J, Hyun J. Thermoresponsive pore structure of biopolymer microspheres for a smart drug carrier. Langmuir: the ACS journal of surfaces and colloids. Jul. 6, 2010; 26(13):11165-11169.
67. Blit P H, McClung W G, Brash J L, Woodhouse K A, Santerre J P. Platelet inhibition and endothelial cell adhesion on elastin-like polypeptide surface modified materials. Biomaterials. September 2011; 32(25):5790-5800.
68. Hearst S M, Walker L R, Shao Q, Lopez M, Raucher D, Vig P J. The design and delivery of a thermally responsive peptide to inhibit S100B-mediated neurodegeneration. Neuroscience. Dec. 1, 2011; 197:369-380.
69. Chen T H, Bae Y, Furgeson D Y, Kwon G S. Biodegradable hybrid recombinant block copolymers for non-viral gene transfection. Int Pharm. May 1, 2012; 427(1):105-112.
70. Amruthwar S S, Puckett A D, Janorkar A V. Preparation and characterization of novel elastin-like polypeptide-collagen composites. J Biomed Mater Res A. Feb. 20, 2013.
71. Amruthwar S S, Janorkar A V. In vitro evaluation of elastin-like polypeptide-collagen composite scaffold for bone tissue engineering. Dental materials: official publication of the Academy of Dental Materials. February 2013; 29(2):211-220.
72. Lee K M, Jung G S, Park J K, Choi S K, Jeon W B. Effects of Arg-Gly-Asp-modified elastin-like polypeptide on pseudoislet formation via up-regulation of cell adhesion molecules and extracellular matrix proteins. Acta Biomater. March 2013; 9(3):5600-5608.
73. Amiram M, Luginbuhl K M, Li X, Feinglos M N, Chilkoti A. Injectable protease-operated depots of glucagon-like peptide-1 provide extended and tunable glucose control. Proc Natl Acad Sci USA. Feb. 19, 2013; 110(8):2792-2797.
74. Senger D R, Galli S J, Dvorak A M, Perruzzi C A, Harvey V S, Dvorak H F. Tumor cells secrete a vascular permeability factor that promotes accumulation of ascites fluid. Science. Feb. 25, 1983; 219(4587):983-985.
75. Hayden M S, Ghosh S. Shared principles in NF-kappaB signaling. Cell. Feb. 8, 2008; 132(3):344-362.
76. May M J, D'Acquisto F, Madge L A, Glockner J, Pober J S, Ghosh S. Selective inhibition of NF-kappaB activation by a peptide that blocks the interaction of NEMO with the IkappaB kinase complex. Science. Sep. 1, 2000; 289(5484):1550-1554.
77. Dasgupta S, Jana M, Zhou Y, Fung Y K, Ghosh S, Pahan K. Antineuroinflammatory effect of NF-kappaB essential modifier-binding domain peptides in the adoptive transfer model of experimental allergic encephalomyelitis. J Immunol. Jul. 15, 2004; 173(2):1344-1354.
78. di Meglio P, Ianaro A, Ghosh S. Amelioration of acute inflammation by systemic administration of a cell-permeable peptide inhibitor of NF-kappaB activation. Arthritis Rheum. March 2005; 52(3):951-958.
79. Tas S W, Vervoordeldonk M J, Hajji N, May M J, Ghosh S, Tak P P. Local treatment with the selective IkappaB kinase beta inhibitor NEMO-binding domain peptide ameliorates synovial inflammation. Arthritis Res Ther. 2006; 8(4):R86.
80. Ghosh A, Roy A, Liu X, et al. Selective inhibition of NF-kappaB activation prevents dopaminergic neuronal loss in a mouse model of Parkinson's disease. Proc Natl Acad Sci USA. Nov. 20, 2007; 104(47):18754-18759.
81. Rehman K K, Bertera S, Bottino R, et al. Protection of islets by in situ peptide-mediated transduction of the Ikappa B kinase inhibitor Nemo-binding domain peptide. J Biol Chem. Mar. 14, 2003; 278(11):9862-9868.
82. Thomas R P, Farrow B J, Kim S, May M J, Hellmich M R, Evers B M. Selective targeting of the nuclear factor-kappaB pathway enhances tumor necrosis factor-related apoptosis-inducing ligand-mediated pancreatic cancer cell death. Surgery. August 2002; 132(2):127-134.
83. Biswas D K, Shi Q, Baily S, et al. NF-kappa B activation in human breast cancer specimens and its role in cell proliferation and apoptosis. Proc Natl Acad Sci USA. Jul. 6, 2004; 101(27):10137-10142.
84. Tapia M A, Gonzalez-Navarrete I, Dalmases A, et al. Inhibition of the canonical IKK/NF kappa B pathway sensitizes human cancer cells to doxorubicin. Cell Cycle. Sep. 15, 2007; 6(18):2284-2292.
85. Ianaro A, Tersigni M, Belardo G, et al. NEMO-binding domain peptide inhibits proliferation of human melanoma cells. Cancer Lett. Nov. 10, 2008.
86. Agou F, Courtois G, Chiaravalli J, et al. Inhibition of NF-kappa B activation by peptides targeting NF-kappa B essential modulator (nemo) oligomerization. J Biol Chem. Dec. 24, 2004; 279(52):54248-54257.
87. Carvalho G, Fabre C, Braun T, et al. Inhibition of NEMO, the regulatory subunit of the IKK complex, induces apoptosis in high-risk myelodysplastic syndrome and acute myeloid leukemia. Oncogene. Apr. 5, 2007; 26(16):2299-2307.
88. Yaron A, Gonen H, Alkalay I, et al. Inhibition of NF-kappa-B cellular function via specific targeting of the I-kappa-B-ubiquitin ligase. Embo J Nov. 3, 1997; 16(21):6486-6494.
89. Takada Y, Singh S, Aggarwal B B. Identification of a p65 peptide that selectively inhibits NF-kappa B activation induced by various inflammatory stimuli and its role in down-regulation of NF-kappaB-mediated gene expression and up-regulation of apoptosis. J Biol Chem. Apr. 9, 2004; 279(15):15096-15104.
90. Torgerson T R, Colosia A D, Donahue J P, Lin Y Z, Hawiger J. Regulation of NF-kappa B, AP-1, NFAT, and STAT1 nuclear import in T lymphocytes by noninvasive delivery of peptide carrying the nuclear localization sequence of NF-kappa B p50. J Immunol. Dec. 1, 1998; 161(11):6084-6092.
91. Letoha T, Somlai C, Takacs T, et al. A nuclear import inhibitory peptide ameliorates the severity of cholecystokinin-induced acute pancreatitis. World J Gastroenterol. Feb. 21, 2005; 11(7):990-999.
92. Saika S, Miyamoto T, Yamanaka 0, et al. Therapeutic effect of topical administration of SN50, an inhibitor of nuclear factor-kappaB, in treatment of corneal alkali burns in mice. Am J Pathol. May 2005; 166(5):1393-1403.
93. Gonzalez-Ramos R, Van Langendonckt A, Defrere S, et al. Agents blocking the nuclear factor-kappaB pathway are effective inhibitors of endometriosis in an in vivo experimental model. Gynecol Obstet Invest. 2008; 65(3):174-186.
94. Poulaki V, Mitsiades C S, Joussen A M, Lappas A, Kirchhof B, Mitsiades N. Constitutive nuclear factor-kappaB activity is crucial for human retinoblastoma cell viability. Am J Pathol. December 2002; 161(6):2229-2240.
95. Starenki D, Namba H, Saenko V, Ohtsuru A, Yamashita S. Inhibition of nuclear factor-kappaB cascade potentiates the effect of a combination treatment of anaplastic thyroid cancer cells. J Clin Endocrinol Metab. January 2004; 89(1):410-418.
96. Xu Y, Fang F, St Clair D K, Sompol P, Josson S, St Clair W H. SN52, a novel nuclear factor-kappaB inhibitor, blocks nuclear import of RelB:p52 dimer and sensitizes prostate cancer cells to ionizing radiation. Mol Cancer Ther. August 2008; 7(8):2367-2376.
97. Yan Liu X, Robinson D, Veach R A, et al. Peptide-directed suppression of a pro-inflammatory cytokine response. J Biol Chem. Jun. 2, 2000; 275(22):16774-16778.
98. Lambeth J D, Krause K H, Clark R A. NOX enzymes as novel targets for drug development. Seminars in immunopathology. July 2008; 30(3):339-363.
99. Rey F E, Cifuentes M E, Kiarash A, Quinn M T, Pagano P J. Novel competitive inhibitor of NAD(P)H oxidase assembly attenuates vascular O(2)(−) and systolic blood pressure in mice. Circulation research. Aug. 31, 2001; 89(5):408-414.
100. Seymour L W, Duncan R, Strohalm J, Kopecek J. Effect of molecular weight (Mw) of N-(2-hydroxypropyl)methacrylamide copolymers on body distribution and rate of excretion after subcutaneous, intraperitoneal, and intravenous administration to rats. J Biomed Mater Res. November 1987; 21(11):1341-1358.
101. Pasqualini R, Ruoslahti E. Organ targeting in vivo using phage display peptide libraries. Nature. 1996; 380:364-6.
102. Federal Register, Vol. 73, No. 104, May 29, 2008.
103. Postmarket Drug Safety, Information for Patients and Providers, fda.gov/Drugs/DrugSafety/PostmarketDrugSafetyInformationforPatientsandProviders/uc m111085.htm.
104. Nouri F S, Wang X, Chen X, Hatefi A. Reducing the Visibility of the Vector/DNA Nanocomplexes to the Immune System by Elastin-Like Peptides. Pharm Res. September 2015; 32(9):3018-28.
105. Logue O C, Mandi F, Chapman H, George E M, Bidwell G L, 3rd. A Maternally Sequestered, Biopolymer-Stabilized Vascular Endothelial Growth Factor (VEGF) Chimera for Treatment of Preeclampsia. J Am. Heart Assoc. Dec. 8, 2017; 6(12).
106. Eddy A C, Howell J A, Chapman H, Taylor E, Mandi F, George E M, Bidwell G L, 3rd. Biopolymer-Delivered, Maternally Sequestered NF-κB (Nuclear Factor-K B) Inhibitory Peptide for Treatment of Preeclampsia. Hypertension. January 2020; 75(1):193-201.
107. Bidwell G L, 3rd, Mandi F, Shao Q, Logue O C, Waller J P, Reese C, Chade A R. A Kidney-Selective Biopolymer for Targeted Drug Delivery. Am J Physiol Renal Physiol. January 2017; 312(1):F54-F64.
108. Kuna M, Waller J P, Logue O C, Bidwell G L, 3rd. Polymer size affects biodistribution and placental accumulation of the drug delivery biopolymer elastin-like polypeptide in a rodent pregnancy model. Placenta. December 2018; 72-73:20-27.
109. Kuna M, Mandi F, Chade A R, Bidwell G L 3rd. Molecular Size Modulates Pharmacokinetics, Biodistribution, and Renal Deposition of the Drug Delivery Biopolymer Elastin-like Polypeptide. Sci Rep. May 2018; 8(1):7923.

1. A placental region targeting elastin-like polypeptide (ELP) comprising:
between 5 and 671 repeat units having the sequence VPGXG;
wherein X in each of the repeat units is individually selected from the group consisting of any amino acid except proline; and
wherein the placental region targeting ELP is selected from the group consisting of a chorionic plate targeting ELP and a chorionic plate and labyrinth/junctional zones targeting ELP.

2. The ELP of claim 1, wherein the ELP is a chorionic plate targeting ELP.

3. The ELP of claim 2, wherein the ELP comprises up to 70 of the repeat units.

4. The ELP of claim 3, wherein the ELP comprises between 5 and 70 of the repeat units.

5. The ELP of claim 2, wherein the ELP comprises a molecular weight of up to 30 kDa.

6. The ELP of claim 5, wherein the ELP comprises a molecular weight of between 3 kDa and 30 kDa.

7. The ELP of claim 2, wherein at least 90% of the ELP accumulates in the chorionic plate.

8. The ELP of claim 1, wherein the ELP is a chorionic plate and labyrinth/junctional zones targeting ELP.

9. The ELP of claim 8, wherein the ELP comprises at least 95 of the repeat units.

10. The ELP of claim 9, wherein the ELP comprises between 95 and 671 of the repeat units.

11. The ELP of claim 8, wherein the ELP comprises a molecular weight of at least 37 kDa.

12. The ELP of claim 10, wherein the ELP comprises a molecular weight of between 37 kDa and 257 kDa.

13. The ELP of claim 8, wherein at least 15% of the ELP accumulates in labyrinth/junctional zones of the placenta.

14. The ELP of claim 1, wherein the repeat units include V:G:A in a 1:4:3 ratio.

15. The ELP of claim 1, further comprising one or more of a group selected from a therapeutic agent or agents, a drug binding domain, a targeting domain, and a cell penetrating peptide.

16. A method of treating a disease in a pregnant subject, the method comprising:
administering a placental region targeting elastin-like peptide (ELP) and a therapeutic drug to a subject in need thereof;
wherein the ELP includes between 5 and 700 repeat units having the sequence VPGXG (SEQ ID NO: 1);
wherein X in each of the repeat units is individually selected from the group consisting of any amino acid except proline; and
wherein the placental region targeting ELP is selected from the group consisting of a chorionic plate targeting ELP, a chorionic plate and labyrinth/junctional zones targeting ELP, and a primarily labyrinth/junctional zones targeting ELP.

17. The method of claim 16, wherein the placental region targeting ELP further comprises one or more of a group selected from a drug binding domain, a targeting domain, and a cell penetrating peptide.

18. A method for decreasing the rate of clearance of an elastin-like polypeptide (ELP) from plasma or a tissue, the method comprising increasing the number of repeat units in the ELP.
